(19) United States
(12) Patent Application Publication
(10) Pub. No.: US 2023/0346970 A1
(43) Pub. Date: Nov. 2, 2023

(54) CENTRAL NERVOUS SYSTEM DELIVERY OF NONSTEROIDAL ANTI-INFLAMMATORY DRUGS AND PSILOCYBIN

(71) Applicant: SILO PHARMA, INC., Englewood Cliffs, NJ (US)

(72) Inventor: Eric Weisblum, Englewood Cliffs, NJ (US)

(21) Appl. No.: 18/004,546

(22) PCT Filed: Jul. 30, 2021
(86) PCT No.: PCT/US2021/044032

§ 371 Date: Jan. 6, 2023

Related U.S. Application Data
(60) Provisional application No. 63/060,577, filed on Aug. 3, 2020.

(51) Int. Cl. A61K 47/69; A61K 47/64; A61K 31/675; A61K 31/616; A61P 25/28; A61P 37/06

(52) U.S. Cl. CPC A61K 47/6911; A61K 47/64; A61K 31/675; A61K 31/616; A61P 25/28; A61P 37/06

(57) ABSTRACT

Disclosed herein include methods, compositions, and kits for treating a disease. In some embodiments, a composition for use in treating a disease comprises a central nervous system homing or targeting peptide associated with a nonsteroidal anti-inflammatory drug (NS AID) and psilocybin or an analogue thereof.

### CROSS-REFERENCE TO RELATED APPLICATIONS

This application claims the benefit under 35 U.S.C. § 119(e) to U.S. Provisional Patent Application No. 63/060,577 filed on Aug. 3, 2020. The content of this related applications is incorporated herein by reference in its entirety for all purposes.

### REFERENCE TO SEQUENCE LISTING

The present application is being filed along with a Sequence Listing in electronic format. The Sequence Listing is provided as a file entitled Sequence_Listing_72GJ-319203-WO, created Jul. 29, 2021, which is 4 kilobytes in size. The information in the electronic format of the Sequence Listing is incorporated herein by reference in its entirety.

### BACKGROUND

### Field

The present disclosure relates generally to the field of disease treatment, for example targeted disease treatment.

### Description of the Related Art

Targeted delivery of a drug, prodrug, or therapeutic agent to cells that cause a disease or are affected by a disease can improve treatment of the disease. There is a need for targeted delivery of drugs, prodrugs, or other therapeutic agents.

### SUMMARY

Disclosed herein include methods of treating a disease in a subject in need thereof. In some embodiments, a method of treating a disease in a subject in need thereof comprises administering to the subject a therapeutically effective amount of a pharmaceutical composition. The pharmaceutical composition can comprise a central nervous system (CNS) homing peptide comprising an amino acid sequence of any one of SEQ ID NOs: 1-22 associated with (i) a nonsteroidal anti-inflammatory drug (NSAID), or a pharmaceutically acceptable salt, co-crystal, polymorph, hydrate, solvate, stereoisomer, or pro-drug thereof, and (ii) psilocybin, or a pharmaceutically acceptable salt, co-crystal, polymorph, hydrate, solvate, stereoisomer, or pro-drug thereof. In some embodiments, a method of treating a disease in a subject in need thereof comprises: co-administering to the subject (i) a therapeutically effective amount of a first pharmaceutical composition comprising a first CNS homing peptide associated with a NSAID, or a pharmaceutically acceptable salt, co-crystal, polymorph, hydrate, solvate, stereoisomer, or pro-drug thereof, and (ii) a therapeutically effective amount of a second pharmaceutical composition comprising a second CNS homing peptide associated with psilocybin, or a pharmaceutically acceptable salt, co-crystal, polymorph, hydrate, solvate, stereoisomer, or pro-drug thereof, the first CNS homing peptide and/or the second CNS homing peptide comprises an amino acid sequence of any one of SEQ ID NOs: 1-22.

The co-administering can comprise administering the therapeutically effective amount of the first pharmaceutical composition and the therapeutically effective amount of the second pharmaceutical composition simultaneously or sequentially. In some embodiments, the administering comprises administering to the subject the therapeutically effective amount of the pharmaceutical composition orally, intravenously, or a combination thereof.

In some embodiments, the first CNS homing peptide and the second CNS homing peptide are identical. In some embodiments, the first CNS homing peptide and the second CNS homing peptide are different. In some embodiments, the CNS homing peptide is directly associated with the NSAID and/or psilocybin, optionally different molecules of the CNS homing peptide are directly associated the NSAID and psilocybin. In some embodiments, the CNS homing peptide is covalently attached with the NSAID and/or psilocybin, optional different molecules of the CNS homing peptide are covalently attached with the NSAID and psilocybin. In some embodiments, the CNS homing peptide is covalently attached with each of the NSAID and/or psilocybin via a saturated or unsaturated, substituted or unsubstituted, straight or branched carbon chain, optionally different molecules of the CNS homing peptide are covalently attached with the NSAID and psilocybin via different molecules of a saturated or unsaturated, substituted or unsubstituted, straight or branched carbon chain. In some embodiments, the CNS homing peptide is conjugated to the NSAID and/or psilocybin, optionally different molecules of the CNS homing peptide are conjugated to the NSAID and psilocybin. In some embodiments, the CNS homing peptide is non-covalently attached with the NSAID and/or psilocybin, optionally different molecules of the CNS homing peptide are non-covalently attached with the NSAID and psilocybin. In some embodiments, the CNS homing peptide is indirectly associated with the NSAID and/or psilocybin.

In some embodiments, the pharmaceutical composition comprises a delivery vehicle comprising the CNS homing peptide on an outer surface of the delivery vehicle. In some embodiments, the delivery vehicle comprises a hydrophilic surface and a hydrophobic volume, and the outer surface of the delivery vehicle comprises a hydrophilic outer surface. In some embodiments, the homing peptide is covalently linked to a saturated or unsaturated, substituted or unsubstituted, straight or branched carbon chain inserted into the hydrophobic volume of the delivery vehicle. In some embodiments, the hydrophobic volume of the delivery vehicle comprises the NSAID and/or psilocybin. In some embodiments, the delivery vehicle encloses the NSAID and/or psilocybin. In some embodiments, the delivery vehicle comprises a surfactant and/or a phospholipid. In some embodiments, the delivery vehicle comprises a micelle, a liposome, a bilayer sheet, or a combination thereof.

The molar ratio of the CNS homing peptide and the NSAID in the pharmaceutical composition can be about 10:1 to about 1:10, and/or the molar ratio of the CNS homing peptide and psilocybin in the pharmaceutical composition is about 10:1 to about 1:10. The weight ratio of the CNS homing peptide and the NSAID in the pharmaceutical composition can be about 10:1 to about 1:10, and/or the weight ratio of the CNS homing peptide and psilocybin in the pharmaceutical composition is about 10:1 to about 1:10. In some embodiments, the molar ratio of the NSAID and psilocybin in the pharmaceutical composition is about 10:1 to about 1:10. In some embodiments, the weight ratio of the NSAID and psilocybin in the pharmaceutical composition is about 10:1 to about 1:10.

The NSAID can comprise a salicylate, a propionic acid derivative, an acetic acid derivative, an enolic acid derivative (oxicam), an anthranilic acid derivative, a fenamic acid, a selective COX-2 inhibitor (coxib), a sulfonanilide, or a combination thereof. In some embodiments, the NSAID comprises aspirin, diflunisal, salicylic acid, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, aceclofenac, bromfenac, nabumetone, piroxicam, meloxicam, tenoxicam, droxicam, lornoxicam, isoxicam, phenylbutazone (bute), mefenamic acid, meclofenamic acid, flufenamic acid, tolfenamic acid, celecoxib, rofecoxib, valdecoxib, parecoxib, lumiracoxib, etoricoxib, firocoxib, nimesulide, clonixin, licofelone, H-harpagide, or a combination thereof. In some embodiments, the NSAID comprises oxaprozin, piroxicam, indomethacin, meloxicam, ketoprofen, sulindac, diflunisal, nabumetone, tolmetin, salsalate, etodolac, fenoprofen, flurbiprofen, ketorolac, meclofenamate, mefenamic acid, or a combination thereof.

The therapeutically effective amount of the pharmaceutical composition can comprise about 1 mg to about 100 mg of the NSAID, and/or the therapeutically effective amount of the pharmaceutical composition comprises about 1 mg to about 100 mg of psilocybin. The therapeutically effective amount can comprise about 1 mg to about 100 mg of the pharmaceutical composition. In some embodiments, the therapeutically effective amount of the pharmaceutical composition comprises about 10 μg to about 3000 μg of the NSAID per kilogram of the body weight of the subject, and/or the therapeutically effective amount of the pharmaceutical composition comprises about 10 μg to about 3000 μg of psilocybin per kilogram of the body weight of the subject. The therapeutically effective amount can comprise about 10 μg to about 3000 μg of the pharmaceutical composition per kilogram of the body weight of the subject. In some embodiments, the amount of the NSAID and/or the amount of psilocybin in the therapeutically effective amount of the pharmaceutical composition comprises about 50% to about 150% of a therapeutically effective amount of the NSAID and/or about 50% to about 150% of a therapeutically effective amount of psilocybin when the NSAID and/or psilocybin is administered in the absence of the CNS homing peptide. In some embodiments, the amount of the NSAID and/or the amount of psilocybin in the therapeutically effective amount of the pharmaceutical composition comprises about 50% to about 150% of a therapeutically effective amount of the NSAID and/or about 50% to about 150% of a therapeutically effective amount of psilocybin when the NSAID and/or psilocybin is administered alone.

The method can generate a desired effect in the subject in about 5 minutes to about 100 minutes. The method can generate a desired effect in the subject in 25% to 75% of the time to generate the desired effect when the NSAID and/or psilocybin is administered to the subject in the absence of the CNS homing peptide. In some embodiments, the method thereby generates a desired effect in the subject in 25% to 75% of the time to generate the desired effect when the NSAID and/or psilocybin is administered to the subject alone. In some embodiments, the desired effect lasts about 1 hour to about 12 hours in the subject. The desired effect can comprise a pain-relieving effect, a psychedelic, or a combination thereof.

In some embodiments, the maximum concentration (Cₘₐₓ) of the NSAID in the blood of the subject is about 2 μg/ml to about 12 μg/ml, and/or the maximum concentration of psilocybin in the blood of the subject is about 2 μg/ml to about 12 μg/ml. In some embodiments, the time (Tₘₐₓ) to reach the maximum concentration of the NSAID in the blood of the subject is about 10 minutes to about 150 minutes, and/or the time to reach the maximum concentration of psilocybin in the blood of the subject is about 10 minutes to about 150 minutes. In some embodiments, the elimination half-life (T_{1/2}) of the NSAID in the blood of the subject is about 20 minutes to about 200 minutes, and/or the elimination half-life of the psilocybin in the blood of the subject is about 20 minutes to about 200 minutes. In some embodiments, the maximum concentration (Cₘₐₓ) of the NSAID in cell(s), tissue(s), organ(s), and/or environment(s) thereof of the CNS of the subject is about 2 μg/ml to about 12 μg/ml, and/or the maximum concentration of the psilocybin in cell(s), tissue(s), organ(s), and/or environment(s) thereof of the CNS of the subject is about 2 μg/ml to about 12 μg/ml. In some embodiments, the time (Tₘₐₓ) to reach the maximum concentration of the NSAID in cell(s), tissue(s), organ(s), and/or environment(s) thereof of the CNS of the subject is about 10 minutes to about 150 minutes, and/or the time to reach the maximum concentration of psilocybin in cell(s), tissue(s), organ(s), and/or environment(s) thereof of the CNS of the subject is about 10 minutes to about 150 minutes. In some embodiments, the elimination half-life (T_{1/2}) of the NSAID in cell(s), tissue(s), organ(s), and/or environment(s) thereof of the CNS of the subject is about 20 minutes to about 200 minutes, and/or the elimination half-life of psilocybin in cell(s), tissue(s), organ(s), and/or environment(s) thereof of the CNS of the subject is about 20 minutes to about 200 minutes. In some embodiments, the maximum concentration (Cₘₐₓ) of the NSAID in cell(s), tissue(s), organ(s), and/or environment(s) thereof of the CNS of the subject is about 50% to about 150% of the maximum concentration of the NSAID in the blood of the subject, and/or the maximum concentration of psilocybin in cell(s), tissue(s), organ(s), and/or environment(s) thereof of the CNS of the subject is about 50% to about 150% of the maximum concentration of psilocybin in the blood of the subject. In some embodiments, the time (Tₘₐₓ) to reach the maximum concentration of the NSAID in cell(s), tissue(s), organ(s), and/or environment(s) thereof of the CNS of the subject is about 100% to about 200% of the time to reach the maximum concentration of the NSAID in the blood of the subject, and/or the time to reach the maximum concentration of psilocybin in cell(s), tissue(s), organ(s), and/or environment(s) thereof of psilocybin of the subject is about 100% to about 200% of the time to reach the maximum concentration of psilocybin in the blood of the subject. In some embodiments, the elimination half-life (T_{1/2}) of the NSAID in cell(s), tissue(s), organ(s), and/or environment(s) thereof of the CNS of the subject is about 100% to about 200% of the elimination half-life of the NSAID in the blood of the subject, and/or the elimination half-life of psilocybin in cell(s), tissue(s), organ(s), and/or environment(s) thereof of the CNS of the subject is about 100% to about 200% of the elimination half-life of the psilocybin in the blood of the subject.

In some embodiments, the maximum concentration (Cₘₐₓ) of the NSAID in cell(s), tissue(s), organ(s), and/or environment(s) thereof of the CNS of the subject is about 50% to about 150% of the maximum concentration of the NSAID in cell(s), tissue(s), organ(s), and/or environment(s) thereof of the CNS of the subject when the NSAID is administered in the absence of the CNS homing peptide and/or when the NSAID and psilocybin are administered in the absence of the CNS homing peptide, and/or the maximum concentration of psilocybin in cell(s), tissue(s), organ(s), and/or environment(s) thereof of the CNS of the subject is about 50% to about 150% of the maximum concentration of psilocybin in cell(s), tissue(s), organ(s), and/or environment(s) thereof of the CNS of the subject when psilocybin is administered in the absence of the CNS homing peptide and/or when the NSAID and psilocybin are administered in the absence of the CNS homing peptide. In some embodiments, the maximum concentration (Cₘₐₓ) of the NSAID in cell(s), tissue(s), organ(s), and/or environment(s) thereof of the CNS of the subject is about 50% to about 150% of the maximum concentration of the NSAID in cell(s), tissue(s), organ(s), and/or environment(s) thereof of the CNS of the subject when the NSAID is administered alone and/or when the NSAID and psilocybin are administered alone, and/or the maximum concentration of psilocybin in cell(s), tissue(s), organ(s), and/or environment(s) thereof of the CNS of the subject is about 50% to about 150% of the maximum concentration of psilocybin in cell(s), tissue(s), organ(s), and/or environment(s) thereof of the CNS of the subject when psilocybin is administered alone and/or when the NSAID and psilocybin are administered alone. In some embodiments, the time (Tₘₐₓ) to reach the maximum concentration of the NSAID in cell(s), tissue(s), organ(s), and/or environment(s) thereof of the CNS of the subject is about 100% to about 200% of the time to reach the maximum concentration of the NSAID in cell(s), tissue(s), organ(s), and/or environment(s) thereof of the CNS of the subject when the NSAID is administered in the absence of the CNS homing peptide and/or when the NSAID and psilocybin are administered in the absence of the CNS homing peptide, and/or the time to reach the maximum concentration of psilocybin in cell(s), tissue(s), organ(s), and/or environment(s) thereof of the CNS of the subject is about 100% to about 200% of the time to reach the maximum concentration of psilocybin in cell(s), tissue(s), organ(s), and/or environment(s) thereof of the CNS of the subject when psilocybin is administered in the absence of the CNS homing peptide and/or when the NSAID and psilocybin are administered in the absence of the CNS homing peptide. In some embodiments, the time (Tₘₐₓ) to reach the maximum concentration of the NSAID in cell(s), tissue(s), organ(s), and/or environment(s) thereof of the CNS of the subject is about 100% to about 200% of the time to reach the maximum concentration of the NSAID in cell(s), tissue(s), organ(s), and/or environment(s) thereof of the CNS of the subject when the NSAID is administered alone and/or when the NSAID and psilocybin are administered alone, and/or the time to reach the maximum concentration of psilocybin in cell(s), tissue(s), organ(s), and/or environment(s) thereof of the CNS of the subject is about 100% to about 200% of the time to reach the maximum concentration of psilocybin in cell(s), tissue(s), organ(s), and/or environment(s) thereof of the CNS of the subject when psilocybin is administered alone and/or when the NSAID and psilocybin are administered alone. In some embodiments, the elimination half-life (T_{1/2}) of the NSAID in cell(s), tissue(s), organ(s), and/or environment(s) thereof of the CNS of the subject is about 100% to about 200% of the elimination half-life of the NSAID in cell(s), tissue(s), organ(s), and/or environment(s) thereof of the CNS of the subject when the NSAID is administered alone and/or when the NSAID and psilocybin are administered alone, and/or the elimination half-life of psilocybin in cell(s), tissue(s), organ(s), and/or environment(s) thereof of the CNS of the subject is about 100% to about 200% of the elimination half-life of psilocybin in cell(s), tissue(s), organ(s), and/or environment(s) thereof of the CNS of the subject when psilocybin is administered alone and/or when the NSAID and psilocybin are administered alone. In some embodiments, the elimination half-life (T_{1/2}) of the NSAID in cell(s), tissue(s), organ(s), and/or environment(s) thereof of the CNS of the subject is about 100% to about 200% of the elimination half-life of the NSAID in cell(s), tissue(s), organ(s), and/or environment(s) thereof of the CNS of the subject when the NSAID is administered in the absence of the CNS homing peptide and/or when the NSAID and psilocybin are administered in the absence of the CNS homing peptide, and/or the elimination half-life of psilocybin in cell(s), tissue(s), organ(s), and/or environment(s) thereof of the CNS of the subject is about 100% to about 200% of the elimination half-life of psilocybin in cell(s), tissue(s), organ(s), and/or environment(s) thereof of the CNS of the subject when psilocybin is administered in the absence of the CNS homing peptide and/or when the NSAID and psilocybin are administered in the absence of the CNS homing peptide.

The cell(s), the tissue(s), and/or the organ(s) of the CNS can comprise damaged and/or inflamed cell(s), tissue(s), and/or organ(s). The cell(s), the tissue(s), and/or the organ(s) of the CNS can comprise the brain, the white matter, the gray matter, the brainstem, the cerebellum, the diencephalon, the cerebrum, the spinal cord, the cranial nerve, cell(s) of any of the preceding, tissue(s) of any of the preceding, or a combination thereof.

In some embodiments, the disease is arthritis. In some embodiments, the arthritis is osteoarthritis, rheumatoid arthritis, gout, pseudo-gout, septic arthritis, ankylosing spondylitis, juvenile idiopathic arthritis, Still's disease, or a combination thereof. In some embodiments, the disease is a central nervous system disease. In some embodiments, the central nervous system disease is a movement disorder, a memory disorder, addiction, attention deficit/hyperactivity disorder (ADHD), autism, bipolar disorder, depression, encephalitis, epilepsy/seizure, migraine, multiple sclerosis, a neurodegenerative disorder, a neuroinflammatory disease, Alzheimer's disease, Huntington's disease, Parkinson's disease, Tourette's syndrome, dystonia, or a combination thereof. In some embodiments, the disease is a neuroinflammatory disease. In some embodiments, the neuroinflammatory disease is Parkinson's disease, Alzheimer's disease, multiple sclerosis, or a combination thereof. In some embodiments, the disease is arthritis, osteoarthritis, rheumatoid arthritis, a neuroinflammatory disease, Parkinson's disease, Alzheimer's disease, multiple sclerosis, Huntington's disease, fibromyalgia, Tourette syndrome, chronic pain, post-traumatic stress disorder, addiction, autism, or a combination thereof.

Disclosed herein include pharmaceutical compositions. In some embodiments, a pharmaceutical composition comprises a CNS homing peptide, the CNS homing peptide comprising an amino acid sequence of any one of SEQ ID NOs: 1-22, associated with (i) a NSAID, or a pharmaceutically acceptable salt, co-crystal, polymorph, hydrate, solvate, stereoisomer, or pro-drug thereof, and (ii) psilocybin, or a pharmaceutically acceptable salt, co-crystal, polymorph, hydrate, solvate, stereoisomer, or pro-drug thereof. In some embodiments, a pharmaceutical composition for use in treating a disease comprising a CNS homing peptide, the CNS homing peptide comprising an amino acid sequence of any one of SEQ ID NOs: 1-22, associated with (i) a NSAID, or a pharmaceutically acceptable salt, co-crystal, polymorph, hydrate, solvate, stereoisomer, or pro-drug thereof and (ii) psilocybin, or a pharmaceutically acceptable salt, co-crystal, polymorph, hydrate, solvate, stereoisomer, or pro-drug thereof. In some embodiments, a pharmaceutical composition comprises a first CNS homing peptide associated with (i) a NSAID, or a pharmaceutically acceptable salt, co-crystal, polymorph, hydrate, solvate, stereoisomer, or pro-drug thereof, and (ii) a second CNS homing peptide associated with psilocybin, or a pharmaceutically acceptable salt, co-crystal, polymorph, hydrate, solvate, stereoisomer, or pro-drug thereof, the first CNS homing peptide and/or the second CNS homing peptide comprises an amino acid sequence of any one of SEQ ID NOs: 1-22. In some embodiments, a pharmaceutical composition for use in treating a disease comprises a first CNS homing peptide associated with (i) a NSAID, or a pharmaceutically acceptable salt, co-crystal, polymorph, hydrate, solvate, stereoisomer, or pro-drug thereof, and (ii) a second CNS homing peptide associated with psilocybin, or a pharmaceutically acceptable salt, co-crystal, polymorph, hydrate, solvate, stereoisomer, or pro-drug thereof, the first CNS homing peptide and/or the second CNS homing peptide comprises an amino acid sequence of any one of SEQ ID NOs: 1-22. In some embodiments, the pharmaceutical composition is formulated for oral administration, intravenous administration, or a combination thereof. Disclosed herein include kits for treating a disease or for use in treating a disease. In some embodiments, a kit comprises any pharmaceutical composition disclosed herein and instructions for using the pharmaceutical composition to treat a disease.

In some embodiments, the first CNS homing peptide and the second CNS homing peptide are identical. In some embodiments, the first CNS homing peptide and the second CNS homing peptide are different. In some embodiments, the CNS homing peptide is directly associated with the NSAID and/or psilocybin, optionally different molecules of the CNS homing peptide are directly associated the NSAID and psilocybin. In some embodiments, the CNS homing peptide is covalently attached with the NSAID and/or psilocybin, optional different molecules of the CNS homing peptide are covalently attached with the NSAID and psilocybin. In some embodiments, the CNS homing peptide is covalently attached with the NSAID and/or psilocybin via a saturated or unsaturated, substituted or unsubstituted, straight or branched carbon chain, optional different molecules of the CNS homing peptide are covalently attached with the NSAID and psilocybin, optionally different molecules of the CNS homing peptide are covalently attached with the NSAID and psilocybin via different molecules of a saturated or unsaturated, substituted or unsubstituted, straight or branched carbon chain. In some embodiments, the CNS homing peptide is conjugated to the NSAID and/or psilocybin, optionally different molecules of the CNS homing peptide are conjugated to the NSAID and psilocybin. In some embodiments, the CNS homing peptide is non-covalently attached with the NSAID and/or psilocybin the NSAID and/or psilocybin, optionally different molecules of the CNS homing peptide are non-covalently attached with the NSAID and psilocybin. In some embodiments, the CNS homing peptide is indirectly associated with the NSAID and/or psilocybin.

The pharmaceutical composition can comprise a delivery vehicle comprising the CNS homing peptide on an outer surface of the delivery vehicle. The delivery vehicle can comprise a hydrophilic surface and a hydrophobic volume, and the outer surface of the delivery vehicle comprises a hydrophilic outer surface. The homing peptide can be covalently linked to a saturated or unsaturated, substituted or unsubstituted, straight or branched carbon chain inserted into the hydrophobic volume of the delivery vehicle. In some embodiments, the hydrophobic volume of the delivery vehicle comprises the NSAID and/or psilocybin. In some embodiments, the delivery vehicle encloses the NSAID and/or psilocybin. In some embodiments, the delivery vehicle comprises a surfactant and/or a phospholipid. In some embodiments, the delivery vehicle comprises a micelle, a liposome, a bilayer sheet, or a combination thereof.

The molar ratio of the CNS homing peptide and the NSAID in the pharmaceutical composition can be about 10:1 to about 1:10, and/or the molar ratio of the CNS homing peptide and psilocybin in the pharmaceutical composition can be about 10:1 to about 1:10. In some embodiments, the weight ratio of the CNS homing peptide and the NSAID in the pharmaceutical composition is about 10:1 to about 1:10, and/or the weight ratio of the CNS homing peptide and psilocybin in the pharmaceutical composition is about 10:1 to about 1:10. In some embodiments, the molar ratio of the NSAID and psilocybin in the pharmaceutical composition is about 10:1 to about 1:10. In some embodiments, the weight ratio of the NSAID and psilocybin in the pharmaceutical composition is about 10:1 to about 1:10.

The NSAID can comprise a salicylate, a propionic acid derivative, an acetic acid derivative, an enolic acid derivative (oxicam), an anthranilic acid derivative, a fenamic acid, a selective COX-2 inhibitor (coxib), a sulfonanilide, or a combination thereof. In some embodiments, the NSAID comprises aspirin, diflunisal, salicylic acid, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, aceclofenac, bromfenac, nabumetone, piroxicam, meloxicam, tenoxicam, droxicam, lornoxicam, isoxicam, phenylbutazone (bute), mefenamic acid, meclofenamic acid, flufenamic acid, tolfenamic acid, celecoxib, rofecoxib, valdecoxib, parecoxib, lumiracoxib, etoricoxib, firocoxib, nimesulide, clonixin, licofelone, H-harpagide, or a combination thereof. In some embodiments, the NSAID comprises oxaprozin, piroxicam, indomethacin, meloxicam, ketoprofen, sulindac, diflunisal, nabumetone, tolmetin, salsalate, etodolac, fenoprofen, flurbiprofen, ketorolac, meclofenamate, mefenamic acid, or a combination thereof.

The pharmaceutical composition can comprise a therapeutically effective amount of about 1 mg to about 100 mg of the NSAID and/or about 1 mg to about 100 mg of psilocybin. In some embodiments, the pharmaceutical composition comprises a therapeutically effective amount of about 1 mg to about 100 mg of the pharmaceutical composition. In some embodiments, the pharmaceutical composition comprises a therapeutically effective amount of about 10 μg to about 3000 μg of the NSAID per kilogram of the body weight of a subject being administered the pharmaceutical composition and/or about 10 μg to about 3000 μg of psilocybin per kilogram of the body weight of a subject being administered the pharmaceutical composition. In some embodiments, the pharmaceutical composition comprises a therapeutically effective amount of about 10 μg to about 3000 μg of the pharmaceutical composition per kilogram of the body weight of a subject being administered the pharmaceutical composition.

In some embodiments, the amount of the NSAID and/or the amount of psilocybin in a therapeutically effective amount of the pharmaceutical composition comprises about 50% to about 150% of a therapeutically effective amount of the NSAID and/or about 50% to about 150% of a therapeutically effective amount of psilocybin when the NSAID and/or psilocybin is administered in the absence of the CNS homing peptide. In some embodiments, the amount of the NSAID and/or the amount of psilocybin in a therapeutically effective amount of the pharmaceutical composition comprises about 50% to about 150% of a therapeutically effective amount of the NSAID and/or about 50% to about 150% of a therapeutically effective amount when the NSAID and/or psilocybin is administered alone.

In some embodiments, the therapeutically effective amount is capable of generating the desired effect in a subject being administered the pharmaceutical composition in about 5 minutes to about 100 minutes. In some embodiments, the therapeutically effective amount is capable of generating a desired effect in a subject being administered the pharmaceutical composition in 25% to 75% of the time to generate the desired effect when the NSAID and/or psilocybin is administered to the subject in the absence of the CNS homing peptide. In some embodiments, the therapeutically effective amount is capable of generating a desired effect in a subject being administered the pharmaceutical composition in 25% to 75% of the time to generate the desired effect when the NSAID and/or psilocybin is administered to the subject alone. In some embodiments, the desired effect lasts about 1 hour to about 12 hours in the subject. In some embodiments, the desired effect comprises a pain-relieving effect, a psychedelic, or a combination thereof.

In some embodiments, the maximum concentration (Cₘₐₓ) of the NSAID in the blood of a subject being administered the pharmaceutical composition is about 2 μg/ml to about 12 μg/ml, and/or the maximum concentration of psilocybin in the blood of the subject is about 2 μg/ml to about 12 μg/ml. In some embodiments, the time (Tₘₐₓ) to reach the maximum concentration of the NSAID in the blood of a subject being administered the pharmaceutical composition is about 10 minutes to about 150 minutes, and/or the time to reach the maximum concentration of psilocybin in the blood of the subject is about 10 minutes to about 150 minutes. In some embodiments, the elimination half-life (T_{1/2}) of the NSAID in the blood of a subject being administered the pharmaceutical composition is about 20 minutes to about 200 minutes, and/or the elimination half-life of psilocybin in the blood of the subject is about 20 minutes to about 200 minutes. In some embodiments, the maximum concentration (Cₘₐₓ) of the NSAID in cell(s), tissue(s), organ(s), and/or environment(s) thereof of the CNS of a subject being administered the pharmaceutical composition is about 2 μg/ml to about 12 μg/ml, and/or the maximum concentration of the psilocybin in cell(s), tissue(s), organ(s), and/or environment(s) thereof of the CNS of the subject is about 2 μg/ml to about 12 μg/ml. In some embodiments, the time (Tₘₐₓ) to reach the maximum concentration of the NSAID in cell(s), tissue(s), organ(s), and/or environment(s) thereof of the CNS of a subject being administered the pharmaceutical composition is about 10 minutes to about 150 minutes, and/or the time to reach the maximum concentration of psilocybin in cell(s), tissue(s), organ(s), and/or environment(s) thereof of the CNS of the subject is about 10 minutes to about 150 minutes. In some embodiments, the elimination half-life (T_{1/2}) of the NSAID in cell(s), tissue(s), organ(s), and/or environment(s) thereof of the CNS of a subject being administered the pharmaceutical composition is about 20 minutes to about 200 minutes, and/or the elimination half-life of psilocybin in cell(s), tissue(s), organ(s), and/or environment(s) thereof of the CNS of the subject is about 20 minutes to about 200 minutes. In some embodiments, the maximum concentration (Cₘₐₓ) of the NSAID in cell(s), tissue(s), organ(s), and/or environment(s) thereof of the CNS of a subject being administered the pharmaceutical composition is about 50% to about 150% of the maximum concentration of the NSAID in the blood of the subject, and/or the maximum concentration of psilocybin in cell(s), tissue(s), organ(s), and/or environment(s) thereof of the CNS of the subject is about 50% to about 150% of the maximum concentration of psilocybin in the blood of the subject. In some embodiments, the time (Tₘₐₓ) to reach the maximum concentration of the NSAID in cell(s), tissue(s), organ(s), and/or environment(s) thereof of the CNS of a subject being administered the pharmaceutical composition is about 100% to about 200% of the time to reach the maximum concentration of the NSAID in the blood of the subject, and/or the time to reach the maximum concentration of psilocybin in cell(s), tissue(s), organ(s), and/or environment(s) thereof of psilocybin of the subject is about 100% to about 200% of the time to reach the maximum concentration of psilocybin in the blood of the subject. In some embodiments, the elimination half-life (T_{1/2}) of the NSAID in cell(s), tissue(s), organ(s), and/or environment(s) thereof of the CNS of a subject being administered the pharmaceutical composition is about 100% to about 200% of the elimination half-life of the NSAID in the blood of the subject, and/or the elimination half-life of psilocybin in cell(s), tissue(s), organ(s), and/or environment(s) thereof of the CNS of the subject is about 100% to about 200% of the elimination half-life of the psilocybin in the blood of the subject.

In some embodiments, the maximum concentration (Cₘₐₓ) of the NSAID in cell(s), tissue(s), organ(s), and/or environment(s) thereof of the CNS of the subject is about 50% to about 150% of the maximum concentration of the NSAID in cell(s), tissue(s), organ(s), and/or environment(s) thereof of the CNS of the subject when the NSAID is administered in the absence of the CNS homing peptide and/or when the NSAID and psilocybin are administered in the absence of the CNS homing peptide, and/or the maximum concentration (Cₘₐₓ) of psilocybin in cell(s), tissue(s), organ(s), and/or environment(s) thereof of the CNS of the subject is about 50% to about 150% of the maximum concentration of psilocybin in cell(s), tissue(s), organ(s), and/or environment(s) thereof of the CNS of the subject when psilocybin is administered in the absence of the CNS homing peptide and/or when the NSAID and psilocybin are administered in the absence of the CNS homing peptide. In some embodiments, the maximum concentration (Cₘₐₓ) of the NSAID in cell(s), tissue(s), organ(s), and/or environment(s) thereof of the CNS of the subject is about 50% to about 150% of the maximum concentration of the NSAID in cell(s), tissue(s), organ(s), and/or environment(s) thereof of the CNS of the subject when the NSAID is administered alone and/or when the NSAID and psilocybin are administered alone, and/or the maximum concentration of psilocybin in cell(s), tissue(s), organ(s), and/or environment(s) thereof of the CNS of the subject is about 50% to about 150% of the maximum concentration of psilocybin in cell(s), tissue(s), organ(s), and/or environment(s) thereof of the CNS of the subject when psilocybin is administered alone and/or when the NSAID and psilocybin are administered alone. In some embodiments, the time (Tₘₐₓ) to reach the maximum concentration of the NSAID in cell(s), tissue(s), organ(s), and/or environment(s) thereof of the CNS of the subject is about 100% to about 200% of the time to reach the maximum concentration of the NSAID in cell(s), tissue(s), organ(s), and/or environment(s) thereof of the CNS of the subject when the NSAID is administered in the absence of the CNS homing peptide and/or when the NSAID and psilocybin are administered in the absence of the CNS homing peptide, and/or the time to reach the maximum concentration of psilocybin in cell(s), tissue(s), organ(s), and/or environment(s) thereof of the CNS of the subject is about 100% to about 200% of the time to reach the maximum concentration of psilocybin in cell(s), tissue(s), organ(s), and/or environment(s) thereof of the CNS of the subject when psilocybin is administered in the absence of the CNS homing peptide and/or when the NSAID and psilocybin are administered in the absence of the CNS homing peptide. In some embodiments, the time (Tₘₐₓ) to reach the maximum concentration of the NSAID in cell(s), tissue(s), organ(s), and/or environment(s) thereof of the CNS of the subject is about 100% to about 200% of the time to reach the maximum concentration of the NSAID in cell(s), tissue(s), organ(s), and/or environment(s) thereof of the CNS of the subject when the NSAID is administered alone and/or when the NSAID and psilocybin are administered alone, and/or the time to reach the maximum concentration of psilocybin in cell(s), tissue(s), organ(s), and/or environment(s) thereof of the CNS of the subject is about 100% to about 200% of the time to reach the maximum concentration of psilocybin in cell(s), tissue(s), organ(s), and/or environment(s) thereof of the CNS of the subject when psilocybin is administered alone and/or when the NSAID and psilocybin are administered alone. In some embodiments, the elimination half-life (T_{1/2}) of the NSAID in cell(s), tissue(s), organ(s), and/or environment(s) thereof of the CNS of the subject is about 100% to about 200% of the elimination half-life of the NSAID in cell(s), tissue(s), organ(s), and/or environment(s) thereof of the CNS of the subject when the NSAID is administered alone and/or when the NSAID and psilocybin are administered alone, and/or the elimination half-life of psilocybin in cell(s), tissue(s), organ(s), and/or environment(s) thereof of the CNS of the subject is about 100% to about 200% of the elimination half-life of psilocybin in cell(s), tissue(s), organ(s), and/or environment(s) thereof of the CNS of the subject when psilocybin is administered alone and/or when the NSAID and psilocybin are administered alone. In some embodiments, the elimination half-life (T_{1/2}) of the NSAID in cell(s), tissue(s), organ(s), and/or environment(s) thereof of the CNS of the subject is about 100% to about 200% of the elimination half-life of the NSAID in cell(s), tissue(s), organ(s), and/or environment(s) thereof of the CNS of the subject when the NSAID is administered in the absence of the CNS homing peptide and/or when the NSAID and psilocybin are administered in the absence of the CNS homing peptide, and/or the elimination half-life of psilocybin in cell(s), tissue(s), organ(s), and/or environment(s) thereof of the CNS of the subject is about 100% to about 200% of the elimination half-life of psilocybin in cell(s), tissue(s), organ(s), and/or environment(s) thereof of the CNS of the subject when psilocybin is administered in the absence of the CNS homing peptide and/or when the NSAID and psilocybin are administered in the absence of the CNS homing peptide.

In some embodiments, the cell(s), the tissue(s), and/or the organ(s) of the CNS comprises damaged and/or inflamed cell(s), tissue(s), and/or organ(s). In some embodiments, the cell(s), the tissue(s), and/or the organ(s) of the CNS comprises the brain, the white matter, the gray matter, the brainstem, the cerebellum, the diencephalon, the cerebrum, the spinal cord, the cranial nerve, cell(s) of any of the preceding, tissue(s) of any of the preceding, or a combination thereof.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the FIGURES, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods of treating a disease in a subject in need thereof. In some embodiments, the method comprises administering to the subject a therapeutically effective amount of a pharmaceutical composition. The pharmaceutical composition can comprise a CNS homing peptide comprising an amino acid sequence of any one of SEQ ID NOs: 1-22 associated with (i) a NSAID, or a pharmaceutically acceptable salt, co-crystal, polymorph, hydrate, solvate, stereoisomer, or pro-drug thereof, and (ii) psilocybin, or a pharmaceutically acceptable salt, co-crystal, polymorph, hydrate, solvate, stereoisomer, or pro-drug thereof. In some embodiments, the method comprises: co-administering to the subject (i) a therapeutically effective amount of a first pharmaceutical composition comprising a first CNS homing peptide associated with a NSAID, or a pharmaceutically acceptable salt, co-crystal, polymorph, hydrate, solvate, stereoisomer, or pro-drug thereof, and (ii) a therapeutically effective amount of a second pharmaceutical composition comprising a second CNS homing peptide associated with psilocybin, or a pharmaceutically acceptable salt, co-crystal, polymorph, hydrate, solvate, stereoisomer, or pro-drug thereof, the first CNS homing peptide and/or the second CNS homing peptide comprises an amino acid sequence of any one of SEQ ID NOs: 1-22.

Disclosed herein include pharmaceutical compositions, comprising a CNS homing peptide, the CNS homing peptide comprising an amino acid sequence of any one of SEQ ID NOs: 1-22, associated with (i) a NSAID, or a pharmaceutically acceptable salt, co-crystal, polymorph, hydrate, solvate, stereoisomer, or pro-drug thereof, and (ii) psilocybin, or a pharmaceutically acceptable salt, co-crystal, polymorph, hydrate, solvate, stereoisomer, or pro-drug thereof. In some embodiments, a pharmaceutical composition for use in treating a disease comprising a CNS homing peptide, the CNS homing peptide comprising an amino acid sequence of any one of SEQ ID NOs: 1-22, associated with (i) a NSAID, or a pharmaceutically acceptable salt, co-crystal, polymorph, hydrate, solvate, stereoisomer, or pro-drug thereof and (ii) psilocybin, or a pharmaceutically acceptable salt, co-crystal, polymorph, hydrate, solvate, stereoisomer, or pro-drug thereof. In some embodiments, a pharmaceutical composition comprises a first CNS homing peptide associated with (i) a NSAID, or a pharmaceutically acceptable salt, co-crystal, polymorph, hydrate, solvate, stereoisomer, or pro-drug thereof, and (i) a second CNS homing peptide associated with psilocybin, or a pharmaceutically acceptable salt, co-crystal, polymorph, hydrate, solvate, stereoisomer, or pro-drug thereof, the first CNS homing peptide and/or the second CNS homing peptide comprises an amino acid sequence of any one of SEQ ID NOs: 1-22. In some embodiments, the pharmaceutical composition comprises a first CNS homing peptide associated with (i) a NSAID, or a pharmaceutically acceptable salt, co-crystal, polymorph, hydrate, solvate, stereoisomer, or pro-drug thereof, and (ii) a second CNS homing peptide associated with psilocybin, or a pharmaceutically acceptable salt, co-crystal, polymorph, hydrate, solvate, stereoisomer, or pro-drug thereof, the first CNS homing peptide and/or the second CNS homing peptide comprises an amino acid sequence of any one of SEQ ID NOs: 1-22. Disclosed herein include kits for treating a disease or for use in treating a disease. In some embodiments, a kit comprises any pharmaceutical composition disclosed herein and instructions for using the pharmaceutical composition to treat a disease.

### Definitions

As used herein, the term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration can be by any suitable route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery of pharmaceutical compositions and therapeutic substances disclosed herein include, but are not limited to, the use of liposomal formulations, intravenous infusion, transdermal patches, or a combination thereof.

As used herein, the term “daily dose” or “daily dosage” refers to a total amount of a pharmaceutical composition or a therapeutic agent that is to be taken within 24 hours.

As used herein, the term “delivery” refers to approaches, formulations, technologies, and systems for transporting a pharmaceutical composition or a therapeutic agent into the body of a patient as needed to safely achieve its desired therapeutic effect. In some embodiments, an effective amount of the composition or agent is formulated for delivery into the blood stream of a patient.

As used herein, the term “formulated” or “formulation” refers to the process in which different chemical substances, including one or more pharmaceutically active ingredients, are combined to produce a dosage form. In some embodiments, two or more pharmaceutically active ingredients can be co-formulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “hydrate” refers to a complex formed by combination of water molecules with molecules or ions of the solute. As used herein, the term “solvate” refers to a complex formed by combination of solvent molecules with molecules or ions of the solute. The solvent can be an organic compound, an inorganic compound, or a mixture of both. Solvate is meant to include hydrate, hemi-hydrate, channel hydrate etc. Some examples of solvents include, but are not limited to, methanol, N,N-dimethylformamide, tetrahydrofuran, dimethylsulfoxide, and water.

As used herein, the term “patient” refers to a subject having a disease. In some embodiments, the patient is a human or an animal. In some embodiments, the patient is a mammal.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to a diseased tissue or a tissue adjacent to the diseased tissue. Carriers or excipients can be used to produce compositions. The carriers or excipients can be chosen to facilitate administration of a drug or pro-drug. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

As used herein, the term “pharmaceutically acceptable salt” refers to any acid or base addition salt whose counter-ions are non-toxic to the patient in pharmaceutical doses of the salts. A host of pharmaceutically acceptable salts are well known in the pharmaceutical field. If pharmaceutically acceptable salts of the compounds of this disclosure are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

The terms “prevent,” “preventing,” “prevention,” and grammatical variations thereof as used herein, refer to a method of partially or completely delaying or precluding the onset or recurrence of a disorder or condition and/or one or more of its attendant symptoms or barring a subject from acquiring or reacquiring a disorder or condition or reducing a subject's risk of acquiring or requiring a disorder or condition or one or more of its attendant symptoms.

As used herein, the term “therapeutically effective” or “effective amount” indicates that a compound or material or amount of the compound or material when administered is sufficient or effective to prevent, alleviate, or ameliorate one or more symptoms of a disease, disorder or medical condition being treated, and/or to prolong the survival of the subject being treated. The therapeutically effective amount will vary depending on the compound, the disease, disorder or condition and its severity and the age, weight, etc., of the mammal to be treated. The dosage can be conveniently administered, e.g., in divided doses up to four times a day or in sustained-release form.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments as described herein may be applied preventively, prophylactically, palliatively or remedially.

### Central Nervous System Delivery of Nonsteroidal Anti-Inflammatory Drugs and Psilocybin or Analogues Thereof

Disclosed herein include methods for treating a disease in a patient or subject in need thereof. In some embodiments, a method of treating a disease in a patient or subject in need thereof comprises administering to the subject a therapeutically effective amount of a pharmaceutical composition (or a pharmaceutically acceptable carrier, pharmaceutically acceptable salt, co-crystal, polymorph, hydrate, or solvate thereof). The pharmaceutical composition can comprise a central nervous system (CNS) homing or targeting peptide. The pharmaceutical composition can comprise a first therapeutic agent or a pharmaceutically acceptable carrier, pharmaceutically acceptable salt, co-crystal, polymorph, hydrate, solvate, stereoisomer, or pro-drug thereof. The therapeutic agent can be nonsteroidal anti-inflammatory drug (NSAID). The CNS homing or targeting peptide can be associated with the therapeutic agent. The pharmaceutical composition can comprise a second therapeutic agent, or a pharmaceutically acceptable carrier, pharmaceutically acceptable salt, co-crystal, polymorph, hydrate, solvate, stereoisomer, or pro-drug of psilocybin or an analogue thereof. The therapeutic agent can comprise psilocybin or an analogue thereof. The CNS homing or targeting peptide can be associated with the second therapeutic agent.

In some embodiments, a method of treating a disease in a patient or subject in need thereof comprises co-administering to the subject therapeutically effective amounts of two pharmaceutical compositions (or a pharmaceutically acceptable carrier, pharmaceutically acceptable salt, co-crystal, polymorph, hydrate, or solvate thereof of each pharmaceutical composition). A first pharmaceutical composition of the two pharmaceutical compositions can comprise a first therapeutic agent (or a pharmaceutically acceptable carrier, pharmaceutically acceptable salt, co-crystal, polymorph, hydrate, solvate, stereoisomer, or pro-drug thereof) associated with a first CNS homing in the first pharmaceutical composition. The first therapeutic agent can be NSAID. The second pharmaceutical composition can comprise a second therapeutic agent (or a pharmaceutically acceptable carrier, pharmaceutically acceptable salt, co-crystal, polymorph, hydrate, solvate, stereoisomer, or pro-drug thereof) associated with a second CNS homing in the second pharmaceutical composition. The second therapeutic agent can comprise psilocybin. In some embodiments, the two CNS homing peptides in the two pharmaceutical compositions can be identical. In some embodiments, the two CNS homing peptides in the two pharmaceutical compositions can be different.

In some embodiments, a pharmaceutical composition can be administered orally. In some embodiments, a pharmaceutical composition can be administered intravenously. In some embodiments, two or more pharmaceutical compositions can be administered separately (e.g., separated by 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 2 weeks, 3 weeks, 4 weeks, 5 weeks, 1 month, 2 months, 3 months, 4 months, 5 months, 6 months, 7 months, 8 months, 9 months, 10 months, 11 months, 12 months, or a number or a range between any two of these values.). In some embodiments, two or more pharmaceutical compositions can be administered simultaneously. For example, the first pharmaceutical composition and the second pharmaceutical composition can be administered to a subject together. In some embodiments, two or more pharmaceutical compositions can be administered sequentially. For example, after the first pharmaceutical composition is administered, the second pharmaceutical composition can be administered. In some embodiments, three or more pharmaceutical compositions can be administered in combination(s). For example, after two pharmaceutical compositions are administered to the subject together, a third pharmaceutical composition can be administered to the subject.

A CNS homing or targeting peptide can comprise, or consist of, one of the amino acid sequences provided in Table 1. In some embodiments, the CNS homing or targeting peptide comprises, or consists of, an amino acid sequence having one, two, three, four, or five amino acid mismatches as compared to one of the amino acid sequences provided in Table 1. In some embodiments, the CNS homing or targeting peptide comprises, or consists of, an amino acid sequence having a deletion of one, two, three, four, or five amino acids as compared to one of the amino acid sequences provided in Table 1. In some embodiments, the CNS homing or targeting peptide comprises, or consists of, an amino acid sequence having one, two, three, four, or five additional amino acids (e.g., additions to the N-terminal, internal additions, and additions to the C-terminal) as compared to one of the amino acid sequences provided in Table 1. CNS homing or targeting peptides have been described in U.S. Patent Application Publication No. 2019/0359651, the content of which is incorporated herein by reference in its entirety.

TABLE 1
CNS homing or targeting peptide sequences

| Amino Acid Sequence | SED ID NO |
|---|---|
| KRSS | 1 |
| PGESS | 2 |
| SLTQ | 3 |
| AMGN | 4 |
| GDRLV | 5 |
| RGGKRSS | 6 |
| SLTQDQG | 7 |
| ADAQADV | 8 |
| RAKGRDA | 9 |
| KASRLGR | 10 |
| RGASMDG | 11 |
| GRPGESS | 12 |
| SRTEGDV | 13 |
| CRGGKRSSC | 14 |
| CSLTQDQGC | 15 |
| CADAQADVC | 16 |
| CRAKGRDAC | 17 |
| CKASRLGRC | 18 |
| CRGASMDGC | 19 |
| CGRPGESSC | 20 |
| CSRTEGDVC | 21 |
| CSGRRGKSC | 22 |

### Pharmaceutical Composition

A pharmaceutical composition (or a pharmaceutically acceptable carrier, pharmaceutically acceptable salt, co-crystal, polymorph, hydrate, or solvate thereof) of the present disclosure can be for use in treating a disease. The pharmaceutical composition can comprise one or more CNS homing peptides. The pharmaceutical composition can comprise one or more therapeutic agents. A combination of two or more pharmaceutical compositions (or a pharmaceutically acceptable carrier, pharmaceutically acceptable salt, co-crystal, polymorph, hydrate, or solvate thereof of each pharmaceutical composition) of the present disclosure can be for use in treating a disease. The two or more pharmaceutical compositions can comprise different CNS homing peptides. The two or more pharmaceutical compositions can comprise different an identical CNS homing peptide. The two or more pharmaceutical compositions can comprise different therapeutic agents. In some embodiments, a pharmaceutical composition is formulated for oral administration, intravenous administration, or a combination thereof. A kit can comprise any pharmaceutical composition disclosed herein and instructions for using the pharmaceutical composition to treat a disease.

Therapeutic Agent and CNS Homing Peptide

In some embodiments, a CNS homing peptide is directly associated with a therapeutic agent (or two or more therapeutic agents). A therapeutic agent can comprise a NSAID (or a pharmaceutically acceptable carrier, pharmaceutically acceptable salt, co-crystal, polymorph, hydrate, solvate, stereoisomer, or pro-drug thereof). A therapeutic agent can comprise psilocybin or an analogue of psilocybin (or a pharmaceutically acceptable carrier, pharmaceutically acceptable salt, co-crystal, polymorph, hydrate, solvate, stereoisomer, or pro-drug thereof). A CNS homing peptide can be covalently attached with a therapeutic agent (or two or more therapeutic agents). A CNS homing peptide can be conjugated to a therapeutic agent (or two or more therapeutic agents). A CNS homing peptide can be covalently attached with a therapeutic agent (or two or more therapeutic agents) via saturated or unsaturated, substituted or unsubstituted, straight or branched carbon chain. A CNS homing peptide can be covalently attached with two or more therapeutic agents via two saturated or unsaturated, substituted or unsubstituted, straight or branched carbon chains. A CNS homing peptide can be non-covalently attached with a therapeutic agent (or two or more therapeutic agents). In some embodiments, a CNS homing peptide is indirectly associated with a therapeutic agent (or two or more therapeutic agents). For example, a pharmaceutical composition comprises a delivery vehicle comprising the CNS homing peptide associated with a therapeutic agent (or two or more therapeutic agents).

In some embodiments, a pharmaceutical composition (or a pharmaceutically acceptable carrier, pharmaceutically acceptable salt, co-crystal, polymorph, hydrate, or solvate thereof) can comprise two or more CNS homing or targeting peptides. In some embodiments, a pharmaceutical composition (or a pharmaceutically acceptable carrier, pharmaceutically acceptable salt, co-crystal, polymorph, hydrate, or solvate thereof) can comprise two or more therapeutic agents, or a pharmaceutically acceptable carrier, pharmaceutically acceptable salt, co-crystal, polymorph, hydrate, solvate, stereoisomer, or pro-drug of each of the two or more therapeutic agents. In some embodiments, one, or each, CNS homing or targeting peptides can be associated with an identical therapeutic agent (e.g., a molecule of a therapeutic agent or different molecules of therapeutic agent). In some embodiments, two, or all, CNS homing or targeting peptides can be associated with different therapeutic agents. In some embodiments, a therapeutic agent in a pharmaceutical composition can be associated with different CNS homing or targeting peptides. In some embodiments, a therapeutic agent in a pharmaceutical composition can be associated with an identical CNS homing or targeting peptide. For example, two therapeutic agents in the pharmaceutical composition are associated with different molecules of a CNS homing or target peptide. For example, a molecule of a therapeutic agent and a molecule of another therapeutic agent in a pharmaceutical composition are associated with a molecule of CNS homing or target peptide.

A pharmaceutical composition can comprise different numbers of CNS homing peptides in different embodiments. In some embodiments, a pharmaceutical composition comprises, comprises about, comprises at least, comprises at least about, comprises at most, or comprises at most about, 1, 2, 3, 4, 5, 6, 7, 8, 9, 10, 11, 12, 13, 14, 15, 16, 17, 18, 19, 20, 30, 40, 50, 60, 70, 80, 90, 100, or a number or a range between any two of these values, CNS homing peptides. Two of the CNS homing peptides (and associated therapeutic agent(s)) can be co-administered simultaneously or sequentially. Two, or more, CNS homing peptides in a pharmaceutical composition can be associated with a therapeutic agent. All CNS homing peptides in a pharmaceutical composition can be associated with molecules of a therapeutic agent. Two, or more, CNS homing peptides in a pharmaceutical composition can be associated with different therapeutic agents. All CNS homing peptides in a pharmaceutical composition can be associated with different therapeutic agents.

A pharmaceutical composition can comprise different numbers of therapeutic agent(s) in different embodiments. In some embodiments, a pharmaceutical composition comprises, comprises about, comprises at least, comprises at least about, comprises at most, or comprises at most about, 1, 2, 3, 4, 5, 6, 7, 8, 9, 10, 11, 12, 13, 14, 15, 16, 17, 18, 19, 20, 30, 40, 50, 60, 70, 80, 90, 100, or a number or a range between any two of these values, therapeutic agents. Two, or more, therapeutic agents can be co-administered simultaneously or sequentially. Two, or more, therapeutic agents in a pharmaceutical composition can be associated with molecules of a CNS homing peptide. All therapeutic agents in a pharmaceutical composition can be associated with molecules of a CNS homing peptide. Two, or more, therapeutic agents in a pharmaceutical composition can be associated with different CNS homing peptides. All therapeutic agents in a pharmaceutical composition can be associated with different CNS homing peptides.

A NSAID in a pharmaceutical composition can be different in different embodiments. A NSAID can comprise a salicylate, a propionic acid derivative, an acetic acid derivative, an enolic acid derivative (e.g., oxicam and derivatives thereof, including piroxicam, isoxicam, meloxicam, tenoxicam, and lornoxicam), an anthranilic acid derivative, a fenamic acid, a selective COX-2 inhibitor (coxib), a sulfonanilide, or a combination thereof. A NSAID can comprise aspirin, diflunisal, salicylic acid, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, aceclofenac, bromfenac, nabumetone, piroxicam, meloxicam, tenoxicam, droxicam, lornoxicam, isoxicam, phenylbutazone (bute), mefenamic acid, meclofenamic acid, flufenamic acid, tolfenamic acid, celecoxib, rofecoxib, valdecoxib, parecoxib, lumiracoxib, etoricoxib, firocoxib, nimesulide, clonixin, licofelone, H-harpagide, or a combination thereof. A NSAID can comprise oxaprozin, piroxicam, indomethacin, meloxicam, ketoprofen, sulindac, diflunisal, nabumetone, tolmetin, salsalate, etodolac, fenoprofen, flurbiprofen, ketorolac, meclofenamate, mefenamic acid, or a combination thereof.

An analogue of psilocybin can have the structure of Formula I:

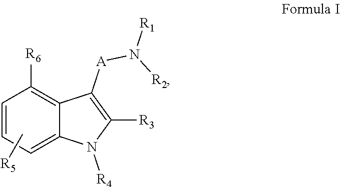

wherein:
A is C₁-C₄ alkylene, C₂-C₄ alkenylene, or C₂-C₄ alkynylene; R₁ and R₂ are, independently for each occurrence, H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen (e.g., F, Cl, Br, I, or At), cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH;
R₃ is H, C₁-C₈ alkyl, C₂-C₈ alkenyl, or C₂-C₈ alkynyl, wherein the C₁-C₈ alkyl, C₂-C₈ alkenyl, or C₂-C₈ alkynyl are optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₃ is selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;
R₄ is H, C₁-C₈ alkyl, C₂-C₈ alkenyl, or C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, and COOH; or R₄ is selected from the group consisting of C₁-C₈ alkylsulfonyl, formyl, hydroxy, C₁-C₈ alkoxyl, —SH, thio(C₂-C₈)alkyl, amino, C₁-C₈ alkylamino, and di(C₁-C₈ alkyl)amino;
R₅ represents 1-3 substituents, each of which is independently selected from the group consisting of C₁-C₈ alkyl, C₂-C₈ alkenyl, and C₂-C₈ alkynyl, each of which is optionally substituted with 1-3 substituents, each of which is independently selected from the group consisting of halogen, cyano, hydroxy, C₁-C₈ alkoxyl, —SH, thio(C₂-C₈)alkyl, amino, C₁-C₈ alkylamino, and di(C₁-C₈ alkyl)amino; or R₅ represents 1-3 substituents, each of which is independently selected from the group consisting of halogen, C₁-C₈ alkylsulfonyl, formyl, COOH, hydroxy, C₁-C₈ alkoxyl, —SH, thio(C₂-C₈)alkyl, amino, C₁-C₈ alkylamino, and di(C₁-C₈ alkyl)amino;
R₆ is phosphate, halogen, R₇ or OR₇; and
R₇ is C₁-C₈ alkyl, optionally substituted with 1-3 substituents, each of which is independently selected from the group consisting of halogen, cyano, hydroxy, oxo, C₁-C₈ alkoxyl, —SH, thio(C₂-C₈)alkyl, amino, C₁-C₈ alkylamino, di(C₁-C₈ alkyl)amino, C₁-C₈ alkylsulfonyl, formyl, COOH, and phosphate; or a pharmaceutically acceptable salt thereof. Analogues of psilocybin have been described in U.S. Patent Application Publication No. 2009/0318527, the content of which is incorporated herein by reference in its entirety.

Weight and Weight Ratio

The weight of a pharmaceutical composition can be different in different embodiments. In some embodiments, the weight of a pharmaceutical composition is, is about, is at least, is at least about, is at most, or is at most about, 10 μg, 20 μg, 30 μg, 40 μg, 50 μg, 60 μg, 70 μg, 80 μg, 90 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 550 mg, 600 mg, 650 mg, 700 mg, 750 mg, 800 mg, 850 mg, 900 mg, 950 mg, 1000 mg, 1 g, 2 g, 3 g, 4 g, 5 g, 6 g, 7 g, 8 g, 9 g, 10 g, or a number or a range between any two of these values. For example, the weight of a pharmaceutical composition is about 1 mg to 100 mg.

The weight of a therapeutic agent in a pharmaceutical composition disclosed herein (or the total weight of two or more therapeutic agents, such as all therapeutic agents, in a pharmaceutical composition) can be different in different embodiments. In some embodiments, the weight of a therapeutic agent (or the total weight of two or more therapeutic agents, such as all therapeutic agents) in a pharmaceutical composition is, is about, is at least, is at least about, is at most, or is at most about, 10 μg, 20 μg, 30 μg, 40 μg, 50 μg, 60 μg, 70 μg, 80 μg, 90 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 550 mg, 600 mg, 650 mg, 700 mg, 750 mg, 800 mg, 850 mg, 900 mg, 950 mg, 1000 mg, 1 g, 2 g, 3 g, 4 g, 5 g, 6 g, 7 g, 8 g, 9 g, 10 g, or a number or a range between any two of these values. For example, a pharmaceutical composition comprises about 1 mg to about 100 mg of a therapeutic agent.

The molar ratio of a CNS homing peptide and a therapeutic agent in a pharmaceutical composition disclosed herein (or the molar ratio of a CNS homing peptide and two or more therapeutic agents, such as all therapeutic agents, in a pharmaceutical composition) can be different in different embodiments. In some embodiments, the molar ratio of a CNS homing peptide and a therapeutic agent in a pharmaceutical composition (or the molar ratio of a CNS homing peptide and two or more therapeutic agents, such as all therapeutic agents, in a pharmaceutical composition) is, is about, is at least, is at least about, is at most, or is at most about, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of these values. For example, the molar ratio of a CNS homing peptide and a NSAID in a pharmaceutical composition is about 10:1 to about 1:10, and/or the molar ratio of a CNS homing peptide and psilocybin in a pharmaceutical composition is about 10:1 to about 1:10.

The weight ratio of a CNS homing peptide and a therapeutic agent in a pharmaceutical composition disclosed herein (or the weight ratio of a CNS homing peptide and two or more therapeutic agents, such as all therapeutic agents, in a pharmaceutical composition) can be different in different embodiments. In some embodiments, the weight ratio of a CNS homing peptide and a therapeutic agent in a pharmaceutical composition (or the weight ratio of a CNS homing peptide and two or more therapeutic agents, such as all therapeutic agents, in a pharmaceutical composition) is, is about, is at least, is at least about, is at most, or is at most about, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of these values. For example, the weight ratio of a CNS homing peptide and a NSAID in a pharmaceutical composition is about 10:1 to about 1:10, and/or the weight ratio of a CNS homing peptide and psilocybin in a pharmaceutical composition is about 10:1 to about 1:10.

The molar ratio of one therapeutic agent and another therapeutic agent (or the molar ratio of one therapeutic agent and the total of two or more therapeutic agents, such as all remaining therapeutic agents) in a pharmaceutical composition disclosed herein can be different in different embodiments. In some embodiments, the molar ratio of one therapeutic agent and another therapeutic agent (or the molar ratio of one therapeutic agent and the total of two or more therapeutic agents, such as all remaining therapeutic agents) in a pharmaceutical composition is, is about, is at least, is at least about, is at most, or is at most about, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of these values. For example, In some embodiments, the molar ratio of a NSAID and psilocybin in a pharmaceutical composition is about 10:1 to about 1:10.

The weight ratio of one therapeutic agent and another therapeutic agent (or the weight ratio of one therapeutic agent and the total of two or more therapeutic agents, such as all remaining therapeutic agents) in a pharmaceutical composition disclosed herein can be different in different embodiments. In some embodiments, the weight ratio of one therapeutic agent and another therapeutic agent (or the weight ratio of one therapeutic agent and the total of two or more therapeutic agents, such as all remaining therapeutic agents) in a pharmaceutical composition is, is about, is at least, is at least about, is at most, or is at most about, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of these values. For example, the weight ratio of a NSAID and psilocybin in a pharmaceutical composition is about 10:1 to about 1:10.

Delivery Vehicle

In some embodiments, a pharmaceutical composition comprises a delivery vehicle. The delivery vehicle can comprise a CNS homing peptide (or two or more CNS homing peptides, such as 2, 3, 4, 5, 6, 7, 8, 9, 10, or more, CNS homing peptides). The CNS homing peptide can be on an outer surface of the delivery vehicle. The delivery vehicle can comprise a hydrophilic surface and a hydrophobic volume. The outer surface of the delivery vehicle can comprise a hydrophilic surface. In some embodiments, the CNS homing peptide is covalently linked to a saturated or unsaturated, substituted or unsubstituted, straight or branched carbon chain inserted into the hydrophobic volume of the delivery vehicle. The hydrophobic volume of the delivery vehicle can comprise a therapeutic agent. The delivery vehicle can enclose or encircle a therapeutic agent. In some embodiments, the delivery vehicle comprises a surfactant. The delivery vehicle can comprise a phospholipid. The delivery vehicle comprises a micelle, a liposome, a bilayer sheet, or a combination thereof.

Carbon Chain

The length of a carbon chain (such as the carbon chain covalently attached to a CNS homing peptide and a therapeutic agent, or the carbon chain covalently attached to a CNS homing peptide and inserted into the hydrophobic volume of a delivery vehicle) can be different in different embodiments. In some embodiments, the length of a carbon chain is, is about, is at least, is at least about, is at most, or is at most about, 10 Å, 11 Å, 12 Å, 13 Å, 14 Å, 15 Å, 16 Å, 17 Å, 18 Å, 19 Å, 20 Å, 21 Å, 22 Å, 23 Å, 24 Å, 25 Å, 26 Å, 27 Å, 28 Å, 29 Å, 30 Å, 31 Å, 32 Å, 33 Å, 34 Å, 35 Å, 36 Å, 37 Å, 38 Å, 39 Å, 40 Å, 41 Å, 42 Å, 43 Å, 44 Å, 45 Å, 46 Å, 47 Å, 48 Å, 49 Å, 50 Å, 51 Å, 52 Å, 53 Å, 54 Å, 55 Å, 56 Å, 57 Å, 58 Å, 59 Å, 60 Å, 61 Å, 62 Å, 63 Å, 64 Å, 65 Å, 66 Å, 67 Å, 68 Å, 69 Å, 70 Å, 71 Å, 72 Å, 73 Å, 74 Å, 75 Å, 76 Å, 77 Å, 78 Å, 79 Å, 80 Å, 81 Å, 82 Å, 83 Å, 84 Å, 85 Å, 86 Å, 87 Å, 88 Å, 89 Å, 90 Å, 91 Å, 92 Å, 93 Å, 94 Å, 95 Å, 96 Å, 97 Å, 98 Å, 99 Å, 100 Å, 200 Å, 300 Å, 400 Å, 500 Å, 600 Å, 700 Å, 800 Å, 900 Å, 1000 Å, or a number or a range between any two of these values.

The carbon chain can comprise one or more carbon atoms, zero, one, or more oxygen atoms, zero, one or more nitrogen atoms, zero, one, or more sulfur atoms, or a combination thereof. In some embodiments, the carbon chain comprises, comprises about, comprises at least, comprises at least about, comprises at most, or comprises at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, or a number or a range between any two of these values, carbon atom(s), oxygen atom(s), nitrogen atom(s), sulfur atom(s), or a combination thereof. The carbon chain can comprise one or more carbon atoms, zero, one, or more oxygen atoms, zero, one or more nitrogen atoms, zero, one, or more sulfur atoms, or a combination thereof, in the main chain of the carbon chain. In some embodiments, the carbon chain comprises, comprises about, comprises at least, comprises at least about, comprises at most, or comprises at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, or a number or a range between any two of these values, carbon atom(s), oxygen atom(s), nitrogen atom(s), sulfur atom(s), or a combination thereof, in the main chain of the carbon chain.

### Therapeutic Efficacy and Pharmacokinetics

Therapeutically Effective Amount

The therapeutically effective amount of a pharmaceutical composition (or a component of a pharmaceutical composition, such as a therapeutic agent) can be the amount of a pharmaceutical composition (or a component of a pharmaceutical composition) administered per time period, per cycle, or per dose. For example, the therapeutically effective amount of a pharmaceutical composition can be the daily dose or dosage. The therapeutically effective amount of a pharmaceutical composition can be administered all in one time (once per time period or cycle) or in several times, such as two times, three times, four times, five times, six times, seven times, eight times, nine times, ten times, or more throughout the time period or cycle.

The therapeutically effective amount of a pharmaceutical composition can be different in different embodiments or different time periods or cycles. For example, the therapeutically effective amount of a pharmaceutical composition of the first cycle and the therapeutically effective amount of subsequent cycles can be different. In some embodiments, the therapeutically effective amount of a pharmaceutical composition is, is about, is at least, is at least about, is at most, or is at most about, 10 μg, 20 μg, 30 μg, 40 μg, 50 μg, 60 μg, 70 μg, 80 μg, 90 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 550 mg, 600 mg, 650 mg, 700 mg, 750 mg, 800 mg, 850 mg, 900 mg, 950 mg, 1000 mg, 1 g, 2 g, 3 g, 4 g, 5 g, 6 g, 7 g, 8 g, 9 g, 10 g, or a number or a range between any two of these values. For example, the therapeutically effective amount of a pharmaceutical composition is about 1 mg to about 100 mg of a pharmaceutical composition.

The number of cycle(s) can be different in different embodiments. In some embodiments, the number of cycle(s) is, is about, is at least, is at least about, is at most, or is at most about, 1, 2, 3, 4, 5, 6, 7, 8, 9, 10, 11, 12, 13, 14, 15, 16, 17, 18, 19, 20, 30, 40, 50, 60, 70, 80, 90, 100, or a number or a range between any two of these values. The time period or the length of a cycle can be different in different embodiments. In some embodiments, the time period or the length of a cycle is, is about, is at least, is at least about, is at most, or is at most about, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 2 weeks, 3 weeks, 4 weeks, 5 weeks, 1 month, 2 months, 3 months, 4 months, 5 months, 6 months, 7 months, 8 months, 9 months, 10 months, 11 months, 12 months, or a number or a range between any two of these values.

The number of dose(s) administered per time period or per cycle, can be different in different embodiments. In some embodiments, the number of dose(s) administered per time period or per cycle is, is about, is at least, is at least about, is at most, or is at most about, 1, 2, 3, 4, 5, 6, 7, 8, 9, 10, 11, 12, 13, 14, 15, 16, 17, 18, 19, 20, 30, 40, 50, 60, 70, 80, 90, 100, or a number or a range between any two of these values. The total number of dose(s) administered can be different in different embodiments. In some embodiments, the total number of dose(s) administered is, is about, is at least, is at least about, is at most, or is at most about, 1, 2, 3, 4, 5, 6, 7, 8, 9, 10, 11, 12, 13, 14, 15, 16, 17, 18, 19, 20, 30, 40, 50, 60, 70, 80, 90, 100, or a number or a range between any two of these values.

The therapeutically effective amount of a pharmaceutical composition can comprise different amounts of a therapeutic agent (or different total amounts of two or more therapeutic agents, such as all therapeutic agents) in a pharmaceutical composition in different embodiments. In some embodiments, the weight of a therapeutic agent (or the total amount of two or more therapeutic agents, or all therapeutic agents) in the therapeutically effective amount of a pharmaceutical composition is, is about, is at least, is at least about, is at most, or is at most about, 10 μg, 20 μg, 30 μg, 40 μg, 50 μg, 60 μg, 70 μg, 80 μg, 90 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 550 mg, 600 mg, 650 mg, 700 mg, 750 mg, 800 mg, 850 mg, 900 mg, 950 mg, 1000 mg, 1 g, 2 g, 3 g, 4 g, 5 g, 6 g, 7 g, 8 g, 9 g, 10 g, or a number or a range between any two of these values. For example, the therapeutically effective amount of a pharmaceutical composition comprises about 1 mg to about 100 mg of a NSAID and/or about 1 mg to about 100 mg of psilocybin (or an analogue of psilocybin).

The therapeutically effective amount of a pharmaceutical composition can be different in different embodiments or different time periods or cycles. For example, the therapeutically effective amount of a pharmaceutical composition comprises about 10 μg to about 3000 μg of the pharmaceutical composition per kilogram of the body weight of the subject (μg/kg) being administered the pharmaceutical composition. In some embodiments, the therapeutically effective amount of a pharmaceutical composition comprises, comprises about, comprises at least, comprises at least about, comprises at most, or comprises at most about, 100 ng/kg, 200 ng/kg, 300 ng/kg, 400 ng/kg, 500 ng/kg, 600 ng/kg, 700 ng/kg, 800 ng/kg, 900 ng/kg, 1000 ng/kg, 2 μg/kg, 3 μg/kg, 4 μg/kg, 5 μg/kg, 6 μg/kg, 7 μg/kg, 8 μg/kg, 9 μg/kg, 10 μg/kg, 20 μg/kg, 30 μg/kg, 40 μg/kg, 50 μg/kg, 60 μg/kg, 70 μg/kg, 80 μg/kg, 90 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1000 μg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 6 mg/kg, 7 mg/kg, 8 mg/kg, 9 mg/kg, 10 mg/kg, or a number or a range between any two of these values, of the pharmaceutical composition.

The therapeutically effective amount of a pharmaceutical composition can comprise different amounts of a therapeutic agent (or different amounts or different total amounts of two or more therapeutic agents, such as all therapeutic agents) in a pharmaceutical composition in different embodiments. For example, the therapeutically effective amount of a pharmaceutical composition comprises about 10 μg to about 3000 μg of a NSAID and/or about 100 μg to about 3000 μg of psilocybin per kilogram of the body weight of the subject (μg/kg) being administered a pharmaceutical composition. In some embodiments, the weight of a therapeutic agent (or the total weight of two or more therapeutic agents, such as all therapeutic agents) in the therapeutically effective amount of a pharmaceutical composition is, is about, is at least, is at least about, is at most, or is at most about, 100 ng/kg, 200 ng/kg, 300 ng/kg, 400 ng/kg, 500 ng/kg, 600 ng/kg, 700 ng/kg, 800 ng/kg, 900 ng/kg, 1000 ng/kg, 2 μg/kg, 3 μg/kg, 4 μg/kg, 5 μg/kg, 6 μg/kg, 7 μg/kg, 8 μg/kg, 9 μg/kg, 10 μg/kg, 20 μg/kg, 30 μg/kg, 40 μg/kg, 50 μg/kg, 60 μg/kg, 70 μg/kg, 80 μg/kg, 90 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1000 μg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 6 mg/kg, 7 mg/kg, 8 mg/kg, 9 mg/kg, 10 mg/kg, or a number or a range between any two of these values.

Targeted delivery using a CNS homing peptide can lower the amount of a therapeutic agent administered while achieving a desired concentration in cell(s), tissue(s), and/or organ(s) of the CNS affected by the disease, thus reducing undesired side effects which may arise from higher dosage levels. In some embodiments, the amount of a therapeutic agent administered with targeted delivery using a CNS homing peptide is lowered by, by about, by at least, by at least about, by at most, or by at most about, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or a number or a range between any two of these values.

The amount of a therapeutic agent (or the amounts of or the total amount of two or more therapeutic agents) in the therapeutically effective amount of a pharmaceutical composition can be less than (or the same as, or more than) a therapeutically effective amount of the therapeutic agent (or the amounts of or the total amount of two or more therapeutic agents) when the therapeutic agent (or the amounts of or the total amount of two or more therapeutic agents) is administered in the absence of the CNS homing peptide or when the therapeutic agent is administered alone. In some embodiments, the amount of a therapeutic agent (or the amounts of or the total amount of two or more therapeutic agents) in the therapeutically effective amount of a pharmaceutical composition is, is about, is at least, is at least about, is at most, or is at most about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, or a number or a range between any two of these values, of a therapeutically effective amount of the therapeutic agent (or the amounts of or the total amount of two or more therapeutic agents) when the therapeutic agent (or the amounts of or the total amount of two or more therapeutic agents) is administered in the absence of the CNS homing peptide or when the therapeutic agent is administered alone. For example, the amount of the NSAID and/or the amount of psilocybin in the therapeutically effective amount of the pharmaceutical composition comprises about 50% to about 150% of a therapeutically effective amount of the NSAID and/or about 50% to about 150% of a therapeutically effective amount of psilocybin when the NSAID and/or psilocybin is administered in the absence of the CNS homing peptide. For example, the amount of the NSAID and/or the amount of psilocybin in the therapeutically effective amount of the pharmaceutical composition comprises about 50% to about 150% of a therapeutically effective amount of the NSAID and/or about 50% to about 150% of a therapeutically effective amount of psilocybin when the NSAID and/or psilocybin is administered alone.

Effect

The therapeutically effective amount of a pharmaceutical composition can treat a disease in the subject. The therapeutically effective amount of a pharmaceutical composition can generate (or result in) a desired effect in the subject. For example, the desired effect comprises a pain-relieving effect, a psychedelic effect, or a combination thereof. The therapeutically effective amount of a pharmaceutical composition can generate (or result in) a desired effect in the subject in different amounts of time after the therapeutically effective amount of the pharmaceutical composition is administered in different embodiments. In some embodiments, the therapeutically effective amount of a pharmaceutical composition generates (or results) in a desired effect in the subject in, in about, in at least, in at least about, in at most, or in at most about, 1 min, 2 mins, 3 mins, 4 mins, 5 mins, 6 mins, 7 mins, 8 mins, 9 mins, 10 mins, 11 mins, 12 mins, 13 mins, 14 mins, 15 mins, 16 mins, 17 mins, 18 mins, 19 mins, 20 mins, 21 mins, 22 mins, 23 mins, 24 mins, 25 mins, 26 mins, 27 mins, 28 mins, 29 mins, 30 mins, 31 mins, 32 mins, 33 mins, 34 mins, 35 mins, 36 mins, 37 mins, 38 mins, 39 mins, 40 mins, 41 mins, 42 mins, 43 mins, 44 mins, 45 mins, 46 mins, 47 mins, 48 mins, 49 mins, 50 mins, 51 mins, 52 mins, 53 mins, 54 mins, 55 mins, 56 mins, 57 mins, 58 mins, 59 mins, 60 mins, 70 mins, 80 mins, 90 mins, 100 mins, 110 mins, 120 mins, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, or a number or a range between any two of these values after the therapeutically effective amount of a pharmaceutical composition is administered to the subject. For example, therapeutically effective amount of a pharmaceutical composition generates a desired effect in the subject in about 5 minutes to about 100 minutes.

Targeted delivery of a therapeutic agent using a CNS homing peptide can shorten delivery time and/or response time. In some embodiments, targeted delivery of a therapeutic agent using a CNS homing peptide can shorten delivery time and/or response by, by about, by at least, by at least about, by at most, or by at most about, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or a number or a range between any two of these values.

The therapeutically effective amount of a pharmaceutical composition can generate (or result in) a desired effect in the subject in less time than (or the same amount of time as, or more than than) the time to generate the desired effect when a therapeutic agent (or two or more therapeutic agents) in the pharmaceutical composition is administered to the subject in the absence of the CNS homing peptide or administered to the subject alone (or by themselves). In some embodiments, the therapeutically effective amount of a pharmaceutical composition generates a desired effect in the subject in 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or a number or a range between any two of these values, of the time needed to generate the desired effect if a therapeutic agent (or two or more therapeutic agents) in the pharmaceutical composition is administered to the subject in the absence of the CNS homing peptide or administered to the subject alone (or by themselves). For example, the therapeutically effective amount of a pharmaceutical composition generates a desired effect in the subject in 25% to 75% of the time needed to generate the desired effect when a NSAID and/or psilocybin in the pharmaceutical composition is administered to the subject in the absence of the CNS homing peptide. For example, the therapeutically effective amount of a pharmaceutical composition generates a desired effect in the subject in 25% to 75% of the time needed to generate the desired effect when the NSAID and/or psilocybin in the pharmaceutical composition is administered to the subject alone (or by themselves)d.

The duration of the desired effect is different in different embodiments. In some embodiments, the duration of the desired effect is, is about, is at least, is at least about, is at most, or is at most about, 10 mins, 20 mins, 30 mins, 40 mins, 50 mins, 60 mins, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, 2 days, 3 days, 4 days, 5 days, 6 days, 7 days, or a number or a range between any two of these values. In some embodiments, the desired effect lasts about 1 hour to about 12 hours in the subject.

Pharmacokinetics

Targeted delivery using a CNS homing peptide can lower the concentration of a therapeutic agent in the bloodstream of the subject while achieving a desired concentration in cell(s), tissue(s), and/or organ(s) of the CNS affected by the disease, thus reducing undesired side effects which may arise from higher dosage levels. In some embodiments, targeted delivery using a CND homing peptide can lower the concentration of a therapeutic agent in the bloodstream of the subject by, by about, by at least, by at least about, by at most, or by at most about, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or a number or a range between any two of these values, lower.

The maximum concentration (Cₘₐₓ) of a therapeutic agent in the blood of the subject can be different in different embodiments. In some embodiments, the maximum concentration of a therapeutic agent in the blood of the subject is, is about, is at least, is at least about, is at most, or is at most about, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 150 ng/ml, 200 ng/ml, 250 ng/ml, 300 ng/ml, 350 ng/ml, 400 ng/ml, 450 ng/ml, 500 ng/ml, 550 ng/ml, 600 ng/ml, 650 ng/ml, 700 ng/ml, 750 ng/ml, 800 ng/ml, 850 ng/ml, 900 ng/ml, 950 ng/ml, 1000 ng/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 21 μg/ml, 22 μg/ml, 23 μg/ml, 24 μg/ml, 25 μg/ml, 26 μg/ml, 27 μg/ml, 28 μg/ml, 29 μg/ml, 30 μg/ml, 31 μg/ml, 32 μg/ml, 33 μg/ml, 34 μg/ml, 35 μg/ml, 36 μg/ml, 37 μg/ml, 38 μg/ml, 39 μg/ml, 40 μg/ml, 41 μg/ml, 42 μg/ml, 43 μg/ml, 44 μg/ml, 45 μg/ml, 46 μg/ml, 47 μg/ml, 48 μg/ml, 49 μg/ml, 50 μg/ml, 60 μg/ml, 70 μg/ml, 80 μg/ml, 90 μg/ml, 100 μg/ml, of a number or a range between any two of these values. For example, the maximum concentration of a NSAID and/or the maximum concentration of psilocybin (or an analogue of psilocybin) in the blood of the subject is about 2 μg/ml to about 12 μg/ml.

The time (Tₘₐₓ) to reach the maximum concentration of a therapeutic agent in the blood of the subject can be different in different subjects. In some embodiments, the time to reach the maximum concentration of a therapeutic agent in the blood of the subject is, is about, is at least, is at least about, is at most, or is at most about, 1 min, 2 mins, 3 mins, 4 mins, 5 mins, 6 mins, 7 mins, 8 mins, 9 mins, 10 mins, 11 mins, 12 mins, 13 mins, 14 mins, 15 mins, 16 mins, 17 mins, 18 mins, 19 mins, 20 mins, 21 mins, 22 mins, 23 mins, 24 mins, 25 mins, 26 mins, 27 mins, 28 mins, 29 mins, 30 mins, 31 mins, 32 mins, 33 mins, 34 mins, 35 mins, 36 mins, 37 mins, 38 mins, 39 mins, 40 mins, 41 mins, 42 mins, 43 mins, 44 mins, 45 mins, 46 mins, 47 mins, 48 mins, 49 mins, 50 mins, 51 mins, 52 mins, 53 mins, 54 mins, 55 mins, 56 mins, 57 mins, 58 mins, 59 mins, 60 mins, 70 mins, 80 mins, 90 mins, 100 mins, 110 mins, 120 mins, 130 mins, 140 mins, 150 mins, 160 mins, 170 mins, 180 mins, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, of a number or a range between any two of these values. For example, the time to reach the maximum concentration of a NSAID and/or the time to reach the maximum concentration of psilocybin (or an analogue of psilocybin) in the blood of the subject is about 10 minutes to about 150 minutes.

The elimination half-life (T_{1/2}) of a therapeutic agent in the blood of the subject can be different in different embodiments. In some embodiments, the elimination half-life of a therapeutic agent in the blood of the subject is, is about, is at least, is at least about, is at most, or is at most about, 1 min, 2 mins, 3 mins, 4 mins, 5 mins, 6 mins, 7 mins, 8 mins, 9 mins, 10 mins, 11 mins, 12 mins, 13 mins, 14 mins, 15 mins, 16 mins, 17 mins, 18 mins, 19 mins, 20 mins, 21 mins, 22 mins, 23 mins, 24 mins, 25 mins, 26 mins, 27 mins, 28 mins, 29 mins, 30 mins, 31 mins, 32 mins, 33 mins, 34 mins, 35 mins, 36 mins, 37 mins, 38 mins, 39 mins, 40 mins, 41 mins, 42 mins, 43 mins, 44 mins, 45 mins, 46 mins, 47 mins, 48 mins, 49 mins, 50 mins, 51 mins, 52 mins, 53 mins, 54 mins, 55 mins, 56 mins, 57 mins, 58 mins, 59 mins, 60 mins, 70 mins, 80 mins, 90 mins, 100 mins, 110 mins, 120 mins, 130 mins, 140 mins, 150 mins, 160 mins, 170 mins, 180 mins, 190 mins, 200 mins, 210 mins, 220 mins, 230 mins, 240 mins, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, or a number or a range between any two of these values. For example, the elimination half-life of a NSAID and/or the elimination half-life of psilocybin (or an analogue of psilocybin) in the blood of the subject is about 20 minutes to about 200 minutes.

The maximum concentration (Cₘₐₓ) of a therapeutic agent in cell(s), tissue(s), organ(s), and/or environment(s) thereof of the CNS of the subject can be different in different embodiments. In some embodiments, the maximum concentration (Cₘₐₓ) of a therapeutic agent in cell(s), tissue(s), organ(s), and/or environment(s) thereof of the CNS of the subject is, is about, is at least, is at least about, is at most, or is at most about, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 150 ng/ml, 200 ng/ml, 250 ng/ml, 300 ng/ml, 350 ng/ml, 400 ng/ml, 450 ng/ml, 500 ng/ml, 550 ng/ml, 600 ng/ml, 650 ng/ml, 700 ng/ml, 750 ng/ml, 800 ng/ml, 850 ng/ml, 900 ng/ml, 950 ng/ml, 1000 ng/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 21 μg/ml, 22 μg/ml, 23 μg/ml, 24 μg/ml, 25 μg/ml, 26 μg/ml, 27 μg/ml, 28 μg/ml, 29 μg/ml, 30 μg/ml, 31 μg/ml, 32 μg/ml, 33 μg/ml, 34 μg/ml, 35 μg/ml, 36 μg/ml, 37 μg/ml, 38 μg/ml, 39 μg/ml, 40 μg/ml, 41 μg/ml, 42 μg/ml, 43 μg/ml, 44 μg/ml, 45 μg/ml, 46 μg/ml, 47 μg/ml, 48 μg/ml, 49 μg/ml, 50 μg/ml, 60 μg/ml, 70 μg/ml, 80 μg/ml, 90 μg/ml, 100 μg/ml, of a number or a range between any two of these values. For example, the maximum concentration of a NSAID and/or the maximum concentration of psilocybin (or an analogue of psilocybin) in cell(s), tissue(s), organ(s), and/or environment(s) thereof of the CNS of the subject is about 2 μg/ml to about 12 μg/ml.

The time (Tₘₐₓ) to reach the maximum concentration of a therapeutic agent in cell(s), tissue(s), organ(s), and/or environment(s) thereof of the CNS of the subject can be different in different embodiments. In some embodiments, the time to reach the maximum concentration of a therapeutic agent in cell(s), tissue(s), organ(s), and/or environment(s) thereof of the CNS of the subject is, is about, is at least, is at least about, is at most, or is at most about, 1 min, 2 mins, 3 mins, 4 mins, 5 mins, 6 mins, 7 mins, 8 mins, 9 mins, 10 mins, 11 mins, 12 mins, 13 mins, 14 mins, 15 mins, 16 mins, 17 mins, 18 mins, 19 mins, 20 mins, 21 mins, 22 mins, 23 mins, 24 mins, 25 mins, 26 mins, 27 mins, 28 mins, 29 mins, 30 mins, 31 mins, 32 mins, 33 mins, 34 mins, 35 mins, 36 mins, 37 mins, 38 mins, 39 mins, 40 mins, 41 mins, 42 mins, 43 mins, 44 mins, 45 mins, 46 mins, 47 mins, 48 mins, 49 mins, 50 mins, 51 mins, 52 mins, 53 mins, 54 mins, 55 mins, 56 mins, 57 mins, 58 mins, 59 mins, 60 mins, 70 mins, 80 mins, 90 mins, 100 mins, 110 mins, 120 mins, 130 mins, 140 mins, 150 mins, 160 mins, 170 mins, 180 mins, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, of a number or a range between any two of these values. For example, the time to reach the maximum concentration of a NSAID and/or the time to reach the maximum concentration of psilocybin (or an analogue of psilocybin) in cell(s), tissue(s), organ(s), and/or environment(s) thereof of the CNS of the subject is about 10 minutes to about 150 minutes.

The elimination half-life (T_{1/2}) of a therapeutic agent in cell(s), tissue(s), organ(s), and/or environment(s) thereof of the CNS of the subject can be different in different embodiments. In some embodiments, the elimination half-life of a therapeutic agent in cell(s), tissue(s), organ(s), and/or environment(s) thereof of the CNS of the subject is, is about, is at least, is at least about, is at most, or is at most about, 1 min, 2 mins, 3 mins, 4 mins, 5 mins, 6 mins, 7 mins, 8 mins, 9 mins, 10 mins, 11 mins, 12 mins, 13 mins, 14 mins, 15 mins, 16 mins, 17 mins, 18 mins, 19 mins, 20 mins, 21 mins, 22 mins, 23 mins, 24 mins, 25 mins, 26 mins, 27 mins, 28 mins, 29 mins, 30 mins, 31 mins, 32 mins, 33 mins, 34 mins, 35 mins, 36 mins, 37 mins, 38 mins, 39 mins, 40 mins, 41 mins, 42 mins, 43 mins, 44 mins, 45 mins, 46 mins, 47 mins, 48 mins, 49 mins, 50 mins, 51 mins, 52 mins, 53 mins, 54 mins, 55 mins, 56 mins, 57 mins, 58 mins, 59 mins, 60 mins, 70 mins, 80 mins, 90 mins, 100 mins, 110 mins, 120 mins, 130 mins, 140 mins, 150 mins, 160 mins, 170 mins, 180 mins, 190 mins, 200 mins, 210 mins, 220 mins, 230 mins, 240 mins, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, or a number or a range between any two of these values. For example, the elimination half-life of a NSAID and/or the elimination half-life of psilocybin (or an analogue of psilocybin) in cell(s), tissue(s), organ(s), and/or environment(s) thereof of the CNS of the subject is about 20 minutes to about 200 minutes.

The maximum concentration (Cₘₐₓ) of a therapeutic agent in cell(s), tissue(s), organ(s), and/or environment(s) thereof of the CNS of the subject can be less than (or the same as, or more than) the maximum concentration of the therapeutic agent in the blood of the subject. In some embodiments, the maximum concentration (Cₘₐₓ) of a therapeutic agent in cell(s), tissue(s), organ(s), and/or environment(s) thereof of the CNS of the subject is, is about, is at least, is at least about, is at most, or is at most about, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, 210%, 220%, 230%, 240%, 250%, 260%, 270%, 280%, 290%, 300%, or a number or a range between any two of these values, of the maximum concentration of the therapeutic agent in the blood of the subject. For example, the maximum concentration of the therapeutic agent and/or the maximum concentration of psilocybin (or an analogue of psilocybin) in cell(s), tissue(s), organ(s), and/or environment(s) thereof of the CNS of the subject is about 50% to about 150% of the maximum concentration of a NSAID and/or the maximum concentration of psilocybin (or an analogue of psilocybin), respectively, in the blood of the subject.

The time (Tₘₐₓ) to reach the maximum concentration of a therapeutic agent in cell(s), tissue(s), organ(s), and/or environment(s) thereof of the CNS of the subject can be less than (or the same as, or more than) the time to reach the maximum concentration of the therapeutic agent in the blood of the subject. In some embodiments, the time to reach the maximum concentration of a therapeutic agent in cell(s), tissue(s), organ(s), and/or environment(s) thereof of the CNS of the subject is, is about, is at least, is at least about, is at most, or is at most about, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, or a number or a range between any two of these values, of the time to reach the maximum concentration of the therapeutic agent in the blood of the subject. For example, the time to reach the maximum concentration of a NSAID and/or the time to reach maximum concentration of psilocybin (or an analogue of psilocybin) in cell(s), tissue(s), organ(s), and/or environment(s) thereof of the CNS of the subject is about 100% to about 200% of the time to reach the maximum concentration of the NSAID and/or the time to reach the maximum concentration of psilocybin (or an analogue of psilocybin), respectively, in the blood of the subject.

The elimination half-life (T_{1/2}) of a therapeutic agent in cell(s), tissue(s), organ(s), and/or environment(s) thereof of the CNS of the subject can be less than (or the same as, or more than) the elimination half-life of the therapeutic agent in the blood of the subject. In some embodiments, the elimination half-life of a therapeutic agent in cell(s), tissue(s), organ(s), and/or environment(s) thereof of the CNS of the subject is, is about, is at least, is at least about, is at most, or is at most about, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, or a number or a range between any two of these values, of the elimination half-life of the therapeutic agent in the blood of the subject. For example, the elimination half-life of a NSAID and/or the elimination half-life of psilocybin (or an analogue of psilocybin) in cell(s), tissue(s), organ(s), and/or environment(s) thereof of the CNS of the subject is about 100% to about 200% of the elimination half-life of the NASID and/or the elimination half-time of psilocybin (or an analogue of psilocybin), respectively, in the blood of the subject.

The maximum concentration (Cₘₐₓ) of a therapeutic agent in cell(s), tissue(s), organ(s), and/or environment(s) thereof of the CNS of the subject can be less than (or the same as, or more than) the maximum concentration of the therapeutic agent in cell(s), tissue(s), organ(s), and/or environment(s) thereof of the CNS of the subject when a therapeutic agent is administered in the absence of the CNS homing peptide, when the therapeutic agent is administered alone, or when the therapeutic agent is administered with another therapeutic agent. In some embodiments, the maximum concentration of a therapeutic agent in cell(s), tissue(s), organ(s), and/or environment(s) thereof of the CNS of the subject is, is about, is at least, is at least about, is at most, or is at most about, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, or a number or a range between any two of these values, of the maximum concentration of the therapeutic agent in cell(s), tissue(s), organ(s), and/or environment(s) thereof of the CNS of the subject when the therapeutic agent is administered in the absence of the CNS homing peptide, when the therapeutic agent is administered alone, or when the therapeutic agent is administered with another therapeutic agent. For example, the maximum concentration of a NSAID and/or the maximum concentration of psilocybin (or an analogue of psilocybin) in cell(s), tissue(s), organ(s), and/or environment(s) thereof of the CNS of the subject is about 50% to about 150% of the maximum concentration of the NSAID and/or the maximum concentration of psilocybin (or an analogue of psilocybin), respectively, in cell(s), tissue(s), organ(s), and/or environment(s) thereof of the CNS of the subject when the NSAID and/or psilocybin (or an analogue of psilocybin) is administered in the absence of the CNS homing peptide. For example, the maximum concentration of a NSAID and/or the maximum concentration of psilocybin (or an analogue of psilocybin) in cell(s), tissue(s), organ(s), and/or environment(s) thereof of the CNS of the subject is about 50% to about 150% of the maximum concentration of the NSAID and/or the maximum concentration of psilocybin (or an analogue of psilocybin), respectively, in cell(s), tissue(s), organ(s), and/or environment(s) thereof of the CNS of the subject when the NSAID and/or psilocybin, or an analogue of psilocybin, is administered alone (or by themselves).

The time (Tₘₐₓ) to reach the maximum concentration of a therapeutic agent in cell(s), tissue(s), organ(s), and/or environment(s) thereof of the CNS of the subject can be less (or the same as, or more than) the time to reach the maximum concentration of the therapeutic agent in cell(s), tissue(s), organ(s), and/or environment(s) thereof of the CNS of the subject when the therapeutic agent is administered in the absence of the CNS homing peptide, when the therapeutic agent is administered alone, or when the therapeutic agent is administered with another therapeutic agent. In some embodiments, the time to reach the maximum concentration of a therapeutic agent in cell(s), tissue(s), organ(s), and/or environment(s) thereof of the CNS of the subject is, is about, is at least, is at least about, is at most, or is at most about, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, or a number or a range between any two of these values, of the time to reach the maximum concentration of the therapeutic agent in cell(s), tissue(s), organ(s), and/or environment(s) thereof of the CNS of the subject when the therapeutic agent is administered in the absence of the CNS homing peptide or when the therapeutic agent is administered alone. For example, the time to reach the maximum concentration of a NSAID and/or the time to reach the maximum concentration of psilocybin (or an analogue of psilocybin) in cell(s), tissue(s), organ(s), and/or environment(s) thereof of the CNS of the subject is about 100% to about 200% of the time to reach the maximum concentration of the NSAID and/or the time to reach the maximum concentration of psilocybin (or an analogue of psilocybin), respectively, in cell(s), tissue(s), organ(s), and/or environment(s) thereof of the CNS of the subject when the NSAID and/or psilocybin (or an analogue of psilocybin) is administered in the absence of the CNS homing peptide. For example, the time to reach the maximum concentration of the NSAID and/or the time to reach the maxim concentration of psilocybin (or an analogue of psilocybin) in cell(s), tissue(s), organ(s), and/or environment(s) thereof of the CNS of the subject is about 100% to about 200% of the time to reach the maximum concentration of the NSAID and/or the time to reach maximum concentration of psilocybin (or an analogue of psilocybin), respectively, in cell(s), tissue(s), organ(s), and/or environment(s) thereof of the CNS of the subject when the NSAID and/or psilocybin (or an analogue of psilocybin) is administered alone (or by themselves).

For example, the elimination half-life (T_{1/2}) of a therapeutic agent in cell(s), tissue(s), organ(s), and/or environment(s) thereof of the CNS of the can be less than (or the same as, or more than) the elimination half-life of the therapeutic agent in cell(s), tissue(s), organ(s), and/or environment(s) thereof of the CNS of the subject when the therapeutic agent is administered in the absence of the CNS homing peptide, when the therapeutic agent is administered alone, or when the therapeutic agent is administered with another therapeutic agent. In some embodiments, the elimination half-life (T_{1/2}) of a therapeutic agent in cell(s), tissue(s), organ(s), and/or environment(s) thereof of the CNS of the subject is, is about, is at least, is at least about, is at most, or is at most about, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, or a number or a range between any two of these values, of the elimination half-life of the therapeutic agent in cell(s), tissue(s), organ(s), and/or environment(s) thereof of the CNS of the subject when the therapeutic agent is administered in the absence of the CNS homing peptide, when the therapeutic agent is administered alone, or when the therapeutic agent is administered with another therapeutic agent. For example, the elimination half-life of a NSAID and/or the elimination half-life of psilocybin (or an analogue of psilocybin) in cell(s), tissue(s), organ(s), and/or environment(s) thereof of the CNS of the subject is about 100% to about 200% of the elimination half-life of the NSAID and/or the elimination half-life of psilocybin (or an analogue of psilocybin), respectively, in cell(s), tissue(s), organ(s), and/or environment(s) thereof of the CNS of the subject when the NSAID and/or psilocybin (or an analogue of psilocybin) is administered in the absence of the CNS homing peptide. For example the elimination half-life of a NSAID and/or the elimination half-life of psilocybin (or an analogue of psilocybin) in cell(s), tissue(s), organ(s), and/or environment(s) thereof of the CNS of the subject is about 100% to about 200% of the elimination half-life of the NSAID and/or the elimination half-life of psilocybin (or an analogue of psilocybin), respectively, in cell(s), tissue(s), organ(s), and/or environment(s) thereof of the CNS of the subject when the NSAID and/or psilocybin, or an analogue of psilocybin, is administered alone (or by themselves).

### Disease

In some embodiments, the cell(s), the tissue(s), and/or the organ(s) of the CNS comprises damaged and/or inflamed cell(s), tissue(s), and/or organ(s). In some embodiments, the cell(s), the tissue(s), and/or the organ(s) of the CNS comprises the brain, the white matter, the gray matter, the brainstem, the cerebellum, the diencephalon, the cerebrum, the spinal cord, the cranial nerve, cell(s) of any of the preceding, tissue(s) of any of the preceding, or a combination thereof.

The therapeutically effective amount of a pharmaceutical composition can treat a disease in the subject. In some embodiments, the disease is arthritis. In some embodiments, the disease is arthritis. In some embodiments, the arthritis is osteoarthritis, rheumatoid arthritis, gout, pseudo-gout, septic arthritis, ankylosing spondylitis, juvenile idiopathic arthritis, Still's disease, or a combination thereof. In some embodiments, the disease is a CNS disease. In some embodiments, the CNS disease is a movement disorder, a memory disorder, addiction, attention deficit/hyperactivity disorder (ADHD), autism, bipolar disorder, depression, encephalitis, epilepsy/seizure, migraine, multiple sclerosis, a neurodegenerative disorder, a neuroinflammatory disease, Alzheimer's disease, Huntington's disease, Parkinson's disease, Tourette syndrome, dystonia, or a combination thereof. In some embodiments, the disease is a neuroinflammatory disease. In some embodiments, the neuroinflammatory disease is Parkinson's disease, Alzheimer's disease, multiple sclerosis, or a combination thereof. In some embodiments, the disease is arthritis, osteoarthritis, rheumatoid arthritis, a neuroinflammatory disease, Parkinson's disease, Alzheimer's disease, multiple sclerosis, Huntington's disease, fibromyalgia, Tourette syndrome, chronic pain, post-traumatic stress disorder, addiction, autism, or a combination thereof.

### Formulation and Administration

Some embodiments provided herein are directed to an effective amount of a pharmaceutical composition comprising a compound (e.g., a therapeutic agent associated with a CNS homing peptide) and at least one pharmaceutically acceptable carrier or excipient. Some embodiments provided herein are directed to a pharmaceutical composition comprising an effective amount of a compound (e.g., a therapeutic agent associated with a CNS homing peptide) and at least one pharmaceutically acceptable carrier or excipient.

Carriers or excipients can be used to produce compositions. The carriers or excipients can be chosen to facilitate administration of the compound or composition. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

Suitable dosage forms, in part, depend upon the use or the route of administration, for example, oral, transdermal, transmucosal, inhalant, or by injection (parenteral). Such dosage forms should allow the compound to reach target cells. Other factors include considerations such as toxicity and dosage forms that retard the compound or composition from exerting its effects.

The compound or composition can be administered by different routes including intravenous, intraperitoneal, subcutaneous, intramuscular, oral, transmucosal, rectal, transdermal, or inhalant. In some embodiments, the composition can be administered by oral administration. For oral administration, for example, the compound or composition can be formulated into conventional oral dosage forms such as capsules, tablets, and liquid preparations such as syrups, elixirs, and concentrated drops.

For inhalants, the compound or composition may be formulated as dry powder or a suitable solution, suspension, or aerosol. Powders and solutions may be formulated with suitable additives. For example, powders may include a suitable powder base such as lactose or starch, and solutions may comprise propylene glycol, sterile water, ethanol, sodium chloride and other additives, such as acid, alkali and buffer salts. Such solutions or suspensions may be administered by inhaling via spray, pump, atomizer, or nebulizer, and the like. The compound or composition may also be used in combination with other inhaled therapies.

Pharmaceutical preparations for oral use can be obtained, for example, by combining the compound with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP, povidone). If desired, disintegrating agents may be added, such as the cross-linked polyvinylpyrrolidone, agar, or alginic acid, or a salt thereof such as sodium alginate.

Dragee cores are provided with suitable coatings. For this purpose, concentrated sugar solutions may be used, which may optionally contain, for example, gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the compound or composition may be dissolved or suspended in suitable liquids, such as fatty oils, liquid paraffin, or liquid polyethylene glycols (PEGs). In addition, stabilizers may be added.

Alternatively, injection (parenteral administration) may be used, e.g., intramuscular, intravenous, intraperitoneal, and/or subcutaneous. For injection, the compound or composition can be formulated in sterile liquid solutions, such as in physiologically compatible buffers or solutions, such as saline solution, Hank's solution, or Ringer's solution. In addition, the compound or composition may be formulated in solid form and redissolved or suspended immediately prior to use. Lyophilized forms can also be produced.

Administration can also be by transmucosal, topical, transdermal, or inhalant means. For transmucosal, topical or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal).

The topical compositions of this disclosure are formulated as oils, creams, lotions, ointments, and the like by choice of appropriate carriers known in the art. Suitable carriers include vegetable or mineral oils, white petrolatum (white soft paraffin), branched chain fats or oils, animal fats and high molecular weight alcohol (greater than C₁₂). In another embodiment, the carriers are those in which the active ingredient is soluble. Emulsifiers, stabilizers, humectants and antioxidants may also be included as well as agents imparting color or fragrance, if desired. Creams for topical application are formulated from a mixture of mineral oil, self-emulsifying beeswax and water in which mixture the active ingredient, dissolved in a small amount solvent (e.g. an oil), is admixed. Additionally, administration by transdermal means may comprise a transdermal patch or dressing such as a bandage impregnated with an active ingredient and optionally one or more carriers or diluents known in the art. To be administered in the form of a transdermal delivery system, the dosage administration will, of course, be continuous rather than intermittent throughout the dosage regimen.

Also provided are kits that include a compound or a pharmaceutical composition thereof. In some embodiments, the compound or composition is packaged, e.g., in a vial, bottle, flask, which may be further packaged, e.g., within a box, envelope, or bag; the compound or composition is approved by the U.S. Food and Drug Administration or similar regulatory agency for administration to a mammal, e.g., a human; the compound or composition is approved for administration to a mammal, e.g., a human, for a CNS disease or condition; the kits described herein may include written instructions for use and/or other indication that the compound or composition is suitable or approved for administration to a mammal, e.g., a human, for a disease or condition as described herein; and the compound or composition may be packaged in unit dose or single dose form, e.g., single dose pills, capsules, or the like.

### EXAMPLES

Some aspects of the embodiments discussed above are disclosed in further detail in the following examples, which are not in any way intended to limit the scope of the present disclosure.

### Example 1

### Peptide-Guided Therapy Using Psilocybin and NSAID

In this example, the pharmacokinetics of psilocybin and a NSAID (e.g., aspirin) delivered to rats having CNS disease via liposomes are evaluated. Psilocybin and a NSAID (e.g., aspirin) are delivered herein via peptide-guided liposomes, which is for enhancing the efficacy and/or toxicity profile of the drugs delivered. As disclosed herein, the peptide-guided liposome delivery can also be adapted for use with other drugs because liposomes can be loaded with both hydrophobic and hydrophilic drugs, thus, permitting combination drug therapy.

Multiple sclerosis (MS) is an autoimmune disease affecting the central nervous system (CNS). Inflammation and demyelination of neurons resulting from a self-directed immune attack leads to muscle weakness and paralysis. Approximately 2.3 million people globally are affected with MS, of which about 1 million reside in the U.S. Many patients manifest a relapsing-remitting pattern of clinical signs and symptoms. Uncontrolled disease can result in severe disability. Despite the availability of several drugs, many MS patients do not respond well to them. In addition, these drugs have many side effects. Thus, there is an urgent need to develop more efficient drug delivery approaches with the purpose of increasing efficacy but reducing adverse effects of these drugs. The currently used drugs are taken orally or given by injection. Therefore, several organs become unintended targets of those drugs that are primarily intended to target the diseased tissue in the CNS. Targeted drug delivery to the CNS can overcome many of the limitations of current therapies. One of the CNS-homing peptides disclosed herein is used as a guide to direct one or more drugs into the CNS and thereby minimize or eliminate off-target side effects. Psilocybin and a NSAID (e.g., aspirin) are incorporated within a nanoparticle, which displays one of the CNS-homing peptide disclosed herein on its surface. The nanoparticle has polyethylene glycol (PEG) on its surface to minimize the otherwise rapid clearance of the particles by the reticuloendothelial system.

Animal Model with Experimental Autoimmune Encephalomyelitis (EAE) for Human MS

Relapsing-remitting disease model in SJL (H-2s) mice (SJL mice), which closely mimics the relapsing nature of MS in humans, is used in this study. In this animal model, proteolipid protein (PLP), a neuronal antigen, is used for disease induction. PLP emulsified in complete Freund's adjuvant (CFA) is injected subcutaneously (s.c.). The onset of signs of EAE and disease progression, including relapses and remissions, are graded on a standardized disease severity scale. Changes in clinical disease are then confirmed by histopathological signs of demyelination in the brain and spinal cord.

### Objective of the Study

Using the EAE model, it is expected that CNS targeting peptide-displaying liposomes can effectively deliver psilocybin to the CNS, and it is superior to plain liposomes or free drug in inhibiting the relapses and progression of EAE. Additionally, specific cell types within the CNS that are targeted by the CNS-homing peptide and CNS-homing peptide-displaying liposomes are expected to be identified.

The CNS-homing peptide-guided drug delivery system is tested for its relative efficacy against relapsing disease in SJL mice compared with that of drug delivery via plain liposomes or as unpackaged (free) drug. To monitor various parameters of systemic toxicity (e.g., toxicity to the liver, kidney, heart, and muscle) in experimental and control groups, the efficacy versus safety profiles of the drug administered via 3 different modalities are compared. The cell types within CNS (e.g., neurons, astrocytes, glial cells, endothelial cells) that are targeted by the CNS homing peptide and CNS homing peptide-displaying liposomes are defined to determine how and where drug-carrying liposomes interact with the CNS tissue cells.

### Experimental Plan

Experiment 1: Pre-clinical efficacy testing in EAE mice. A cohort of SJL mice is injected s.c. with PLP for EAE induction, which appears about d 10 after PLP injection. At the time of onset of EAE, mice are be randomized into 3 sets of two groups each for treatment with psilocybin and the NSAID (e.g., aspirin) administered in three different formulations. A group of untreated EAE mice is served as another control.
(a) Set 1: Peptide-guided liposomal psilocybin delivery. Psilocybin and the NSAID (e.g., aspirin) (Group 1) are entrapped in liposomes. Liposomes containing vehicle only (Group 2) is served as a control. All these liposomes display on their cell surface the CNS-homing peptide as well as polyethylene glycol (PEG); the latter to reduce their clearance by the reticuloendothelial system.
(b) Set 2: liposomes lacking the CNS-homing peptide. Plain liposomes containing psilocybin and the NSAID (e.g., aspirin) (Group 3) or vehicle (Group 4) are prepared.
(c) Set 3: plain psilocybin and the NSAID (e.g., aspirin) (Group 5) or vehicle (Group 6). Set-3 is served as controls for the other two sets.

The liposomes or free drug/vehicles are injected intravenously (iv) into EAE mice. One injection is given at the time of EAE onset, with a second injection after 3 days, and a third injection after another 3 days, coinciding with the peak phase of disease (about d 16 after PLP injection). Throughout this period, the EAE severity of mice is graded in a blinded fashion.

Experiment 2: Histopathological and serological confirmation of disease severity and/or toxicity. After 7-10 days of the third injection, all mice are sacrificed, and various tissues and sera collected are tested as follows: (i) Efficacy: the CNS tissue (brain and spinal cord) are histologically analyzed for signs of perivascular inflammation and demyelination for confirmation of clinical observations; and (ii) Toxicity: the sera are tested for tissue enzymes/markers for toxicity to liver, kidney, pancreas, etc.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof Δny listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of treating a disease in a subject in need thereof, comprising:
administering to the subject a therapeutically effective amount of a first pharmaceutical composition, wherein the pharmaceutical composition comprises a first central nervous system (CNS) homing peptide comprising an amino acid sequence of any one of SEQ ID NOs: 1-22 associated with (i) a nonsteroidal anti-inflammatory drug (NSAID), or a pharmaceutically acceptable salt, co-crystal, polymorph, hydrate, solvate, stereoisomer, or pro-drug thereof, and (ii) psilocybin, or a pharmaceutically acceptable salt, co-crystal, polymorph, hydrate, solvate, stereoisomer, or pro-drug thereof.

2. (canceled)

3. (canceled)

4. (canceled)

5. (canceled)

6. (canceled)

7. (canceled)

8. (canceled)

9. (canceled)

10. The method of claim 1, wherein the CNS homing peptide is indirectly associated with the NSAID and/or psilocybin.

11. (canceled)

12. (canceled)

13. (canceled)

14. (canceled)

15. (canceled)

16. The method of claim 1, wherein the molar ratio or the weight ratio of the CNS homing peptide and the NSAID in the pharmaceutical composition is about 10:1 to about 1:10, and/or wherein the molar ratio or the weight ratio of the CNS homing peptide and psilocybin in the pharmaceutical composition is about 10:1 to about 1:10.

17. (canceled)

18. (canceled)

19. The method of claim 1, wherein the NSAID comprises aspirin, diflunisal, salicylic acid, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, aceclofenac, bromfenac, nabumetone, piroxicam, meloxicam, tenoxicam, droxicam, lornoxicam, isoxicam, phenylbutazone (bute), mefenamic acid, meclofenamic acid, flufenamic acid, tolfenamic acid, celecoxib, rofecoxib, valdecoxib, parecoxib, lumiracoxib, etoricoxib, firocoxib, nimesulide, clonixin, licofelone, H-harpagide, or a combination thereof.

20. (canceled)

21. (canceled)

22. (canceled)

23. (canceled)

24. (canceled)

25. (canceled)

26. (canceled)

27. The method of claim 1, wherein the maximum concentration (Cₘₐₓ) of the NSAID in the blood of the subject is about 2 μg/ml to about 12 μg/ml, and/or wherein the maximum concentration of psilocybin in the blood of the subject is about 2 μg/ml to about 12 μg/ml, and/or wherein the maximum concentration of the NSAID in cell(s), tissue(s), organ(s), and/or environment(s) thereof of the CNS of the subject is about 2 μg/ml to about 12 μg/ml, and/or wherein the maximum concentration of the psilocybin in cell(s), tissue(s), organ(s), and/or environment(s) thereof of the CNS of the subject is about 2 μg/ml to about 12 μg/ml.

28. The method of claim 1, wherein the time (Tₘₐₓ) to reach the maximum concentration of the NSAID in the blood of the subject is about 10 minutes to about 150 minutes, and/or wherein the time to reach the maximum concentration of psilocybin in the blood of the subject is about 10 minutes to about 150 minutes, and/or wherein the time to reach the maximum concentration of the NSAID in cell(s), tissue(s), organ(s), and/or environment(s) thereof of the CNS of the subject is about 10 minutes to about 150 minutes, and/or wherein the time to reach the maximum concentration of psilocybin in cell(s), tissue(s), organ(s), and/or environment(s) thereof of the CNS of the subject is about 10 minutes to about 150 minutes.

29. The method of claim 1, wherein the elimination half-life (T_{1/2}) of the NSAID in the blood of the subject is about 20 minutes to about 200 minutes, and/or wherein the elimination half-life of the psilocybin in the blood of the subject is about 20 minutes to about 200 minutes, and/or the elimination half-life of the NSAID in cell(s), tissue(s), organ(s), and/or environment(s) thereof of the CNS of the subject is about 20 minutes to about 200 minutes, and/or wherein the elimination half-life of psilocybin in cell(s), tissue(s), organ(s), and/or environment(s) thereof of the CNS of the subject is about 20 minutes to about 200 minutes.

30. The method of claim 1, wherein the maximum concentration (Cₘₐₓ) of the NSAID in cell(s), tissue(s), organ(s), and/or environment(s) thereof of the CNS of the subject is about 50% to about 150% of the maximum concentration of the NSAID in the blood of the subject, and/or wherein the maximum concentration of psilocybin in cell(s), tissue(s), organ(s), and/or environment(s) thereof of the CNS of the subject is about 50% to about 150% of the maximum concentration of psilocybin in the blood of the subject.

31. (canceled)

32. (canceled)

33. The method of claim 1,
wherein the maximum concentration (Cₘₐₓ) of the NSAID in cell(s), tissue(s), organ(s), and/or environment(s) thereof of the CNS of the subject is about 50% to about 150% of the maximum concentration of the NSAID in cell(s), tissue(s), organ(s), and/or environment(s) thereof of the CNS of the subject when the NSAID is administered in the absence of the CNS homing peptide and/or when the NSAID and psilocybin are administered in the absence of the CNS homing peptide and/or when the NSAID is administered alone and/or when the NSAID and psilocybin are administered alone, and/or
wherein the maximum concentration of psilocybin in cell(s), tissue(s), organ(s), and/or environment(s) thereof of the CNS of the subject is about 50% to about 150% of the maximum concentration of psilocybin in cell(s), tissue(s), organ(s), and/or environment(s) thereof of the CNS of the subject when psilocybin is administered in the absence of the CNS homing peptide and/or when the NSAID and psilocybin are administered in the absence of the CNS homing peptide and/or when psilocybin is administered alone and/or when the NSAID and psilocybin are administered alone.

34. The method of claim 1,
wherein the time (Tₘₐₓ) to reach the maximum concentration of the NSAID in cell(s), tissue(s), organ(s), and/or environment(s) thereof of the CNS of the subject is about 100% to about 200% of the time to reach the maximum concentration of the NSAID in cell(s), tissue(s), organ(s), and/or environment(s) thereof of the CNS of the subject when the NSAID is administered in the absence of the CNS homing peptide and/or when the NSAID and psilocybin are administered in the absence of the CNS homing peptide and/or when the NSAID is administered alone and/or when the NSAID and psilocybin are administered alone, and/or
wherein the time to reach the maximum concentration of psilocybin in cell(s), tissue(s), organ(s), and/or environment(s) thereof of the CNS of the subject is about 100% to about 200% of the time to reach the maximum concentration of psilocybin in cell(s), tissue(s), organ(s), and/or environment(s) thereof of the CNS of the subject when psilocybin is administered in the absence of the CNS homing peptide and/or when the NSAID and psilocybin are administered in the absence of the CNS homing peptide and/or when psilocybin is administered alone and/or when the NSAID and psilocybin are administered alone.

35. The method of claim 1,
wherein the elimination half-life (T_{1/2}) of the NSAID in cell(s), tissue(s), organ(s), and/or environment(s) thereof of the CNS of the subject is about 100% to about 200% of the elimination half-life of the NSAID in cell(s), tissue(s), organ(s), and/or environment(s) thereof of the CNS of the subject when the NSAID is administered alone and/or when the NSAID and psilocybin are administered alone and/or when the NSAID is administered in the absence of the CNS homing peptide and/or when the NSAID and psilocybin are administered in the absence of the CNS homing peptide, and/or
wherein the elimination half-life of psilocybin in cell(s), tissue(s), organ(s), and/or environment(s) thereof of the CNS of the subject is about 100% to about 200% of the elimination half-life of psilocybin in cell(s), tissue(s), organ(s), and/or environment(s) thereof of the CNS of the subject when psilocybin is administered alone and/or when the NSAID and psilocybin are administered alone and/or when psilocybin is administered in the absence of the CNS homing peptide and/or when the NSAID and psilocybin are administered in the absence of the CNS homing peptide.

36. (canceled)

37. The method of claim 1, wherein the administering comprises administering to the subject the therapeutically effective amount of the pharmaceutical composition orally, intravenously, or a combination thereof.

38. (canceled)

39. (canceled)

40. (canceled)

41. (canceled)

42. A pharmaceutical composition for treatment of a disease in a subject comprising a first central nervous system (CNS) homing peptide associated with (i) a nonsteroidal anti-inflammatory drug (NSAID), or a pharmaceutically acceptable salt, co-crystal, polymorph, hydrate, solvate, stereoisomer, or pro-drug thereof, and (ii) a second CNS homing peptide associated with psilocybin, or a pharmaceutically acceptable salt, co-crystal, polymorph, hydrate, solvate, stereoisomer, or pro-drug thereof, wherein the first CNS homing peptide and/or the second CNS homing peptide comprises an amino acid sequence of any one of SEQ ID NOs: 1-22.

43. (canceled)

44. (canceled)

45. (canceled)

46. (canceled)

47. (canceled)

48. (canceled)

49. The pharmaceutical composition of claim 42, wherein the pharmaceutical composition comprises a delivery vehicle comprising the first CNS homing peptide and the second CNS homing peptide on an outer surface of the delivery vehicle.

50. (canceled)

51. (canceled)

52. (canceled)

53. (canceled)

54. (canceled)

55. (canceled)

56. (canceled)

57. (canceled)

58. (canceled)

59. The pharmaceutical composition of claim 42, comprising a therapeutically effective amount of about 1 mg to about 100 mg of the NSAID and/or about 1 mg to about 100 mg of psilocybin.

60. (canceled)

61. (canceled)

62. (canceled)

63. (canceled)

64. (canceled)

65. The pharmaceutical composition of claim 42, wherein the maximum concentration (Cₘₐₓ) of the NSAID in the blood of a subject being administered the pharmaceutical composition is about 2 μg/ml to about 12 μg/ml, and/or wherein the maximum concentration of psilocybin in the blood of the subject is about 2 μg/ml to about 12 μg/ml, and/or wherein the maximum concentration of the NSAID in cell(s), tissue(s), organ(s), and/or environment(s) thereof of the CNS of a subject being administered the pharmaceutical composition is about 2 μg/ml to about 12 μg/ml, and/or wherein the maximum concentration of the psilocybin in cell(s), tissue(s), organ(s), and/or environment(s) thereof of the CNS of the subject is about 2 μg/ml to about 12 μg/ml.

66. (canceled)

67. (canceled)

68. (canceled)

69. (canceled)

70. (canceled)

71. The pharmaceutical composition of claim 42,
wherein the maximum concentration (Cₘₐₓ) of the NSAID in cell(s), tissue(s), organ(s), and/or environment(s) thereof of the CNS of the subject is about 50% to about 150% of the maximum concentration of the NSAID in cell(s), tissue(s), organ(s), and/or environment(s) thereof of the CNS of the subject when the NSAID is administered in the absence of the CNS homing peptide and/or when the NSAID and psilocybin are administered in the absence of the CNS homing peptide and/or when the NSAID is administered alone and/or when the NSAID and psilocybin are administered alone, and/or
wherein the maximum concentration (Cₘₐₓ) of psilocybin in cell(s), tissue(s), organ(s), and/or environment(s) thereof of the CNS of the subject is about 50% to about 150% of the maximum concentration of psilocybin in cell(s), tissue(s), organ(s), and/or environment(s) thereof of the CNS of the subject when psilocybin is administered in the absence of the CNS homing peptide and/or when the NSAID and psilocybin are administered in the absence of the CNS homing peptide and/or when psilocybin is administered alone and/or when the NSAID and psilocybin are administered alone.

72. (canceled)

73. (canceled)

74. (canceled)

75. (canceled)

76. The pharmaceutical composition of claim 42, wherein (a) the disease is arthritis and optionally the arthritis is osteoarthritis, rheumatoid arthritis, gout, pseudo-gout, septic arthritis, ankylosing spondylitis, juvenile idiopathic arthritis, Still's disease, or a combination thereof; (b) the disease is a central nervous system disease and optionally the central nervous system disease is a movement disorder, a memory disorder, addiction, attention deficit/hyperactivity disorder (ADHD), autism, bipolar disorder, depression, encephalitis, epilepsy/seizure, migraine, multiple sclerosis, a neurodegenerative disorder, a neuroinflammatory disease, Alzheimer's disease, Huntington's disease, Parkinson's disease, Tourette syndrome, dystonia, or a combination thereof; or (c) the disease is a neuroinflammatory disease and optionally the neuroinflammatory disease is Parkinson's disease, Alzheimer's disease, multiple sclerosis, or a combination thereof.

77. The pharmaceutical composition of claim 42, wherein the disease is arthritis, osteoarthritis, rheumatoid arthritis, a neuroinflammatory disease, Parkinson's disease, Alzheimer's disease, multiple sclerosis, Huntington's disease, fibromyalgia, Tourette syndrome, chronic pain, post-traumatic stress disorder, addiction, autism, or a combination thereof.

78. A kit comprising a pharmaceutical composition of claim 42 and instructions for using the pharmaceutical composition to treat a disease.


SEQUENCE LISTING (22 sequences)

SEQ ID NO: 1 - protein, 4 aa, Artificial sequence
  peptide
KRSS

SEQ ID NO: 2 - protein, 5 aa, Artificial sequence
  peptide
PGESS

SEQ ID NO: 3 - protein, 4 aa, Artificial sequence
  peptide
SLTQ

SEQ ID NO: 4 - protein, 4 aa, Artificial sequence
  peptide
AMGN

SEQ ID NO: 5 - protein, 5 aa, Artificial sequence
  peptide
GDRLV

SEQ ID NO: 6 - protein, 7 aa, Artificial sequence
  peptide
RGGKRSS

SEQ ID NO: 7 - protein, 7 aa, Artificial sequence
  peptide
SLTQDQG

SEQ ID NO: 8 - protein, 7 aa, Artificial sequence
  peptide
ADAQADV

SEQ ID NO: 9 - protein, 7 aa, Artificial sequence
  peptide
RAKGRDA

SEQ ID NO: 10 - protein, 7 aa, Artificial sequence
  peptide
KASRLGR

SEQ ID NO: 11 - protein, 7 aa, Artificial sequence
  peptide
RGASMDG

SEQ ID NO: 12 - protein, 7 aa, Artificial sequence
  peptide
GRPGESS

SEQ ID NO: 13 - protein, 7 aa, Artificial sequence
  peptide
SRTEGDV

SEQ ID NO: 14 - protein, 9 aa, Artificial sequence
  peptide
CRGGKRSSC

SEQ ID NO: 15 - protein, 9 aa, Artificial sequence
  peptide
CSLTQDQGC

SEQ ID NO: 16 - protein, 9 aa, Artificial sequence
  peptide
CADAQADVC

SEQ ID NO: 17 - protein, 9 aa, Artificial sequence
  peptide
CRAKGRDAC

SEQ ID NO: 18 - protein, 9 aa, Artificial sequence
  peptide
CKASRLGRC

SEQ ID NO: 19 - protein, 9 aa, Artificial sequence
  peptide
CRGASMDGC

SEQ ID NO: 20 - protein, 9 aa, Artificial sequence
  peptide
CGRPGESSC

SEQ ID NO: 21 - protein, 9 aa, Artificial sequence
  peptide
CSRTEGDVC

SEQ ID NO: 22 - protein, 9 aa, Artificial sequence
  peptide
CSGRRGKSC